(19) United States
(12) Patent Application Publication
(10) Pub. No.: US 2024/0247256 A1
(43) Pub. Date: Jul. 25, 2024

(54) GENE ASSEMBLY FROM OLIGONUCLEOTIDE POOLS

(71) Applicant: CAMENA BIOSCIENCE LIMITED, Essex (GB)

(72) Inventors: Derek L. STEMPLE, Alton, NH (US); Steven HARVEY, Essex (GB); Neil BELL, Essex (GB); James ALLEN, Essex (GB); Ellecia STARR, Essex (GB); Nadin FATHALLAH, Essex (GB)

(21) Appl. No.: 18/560,445

(22) PCT Filed: May 11, 2022
(86) PCT No.: PCT/US2022/028775

§ 371 Date: Nov. 13, 2023

Related U.S. Application Data
(60) Provisional application No. 63/186,871, filed on May 11, 2021.

(51) Int. Cl. C12N 15/11; C12N 9/22

(52) U.S. Cl. CPC C12N 15/11; C12N 9/22; C12N 2310/16

(57) ABSTRACT

Disclosed herein are compositions and methods that utilize high-throughput oligonucleotide synthesis methods, including array-based oligonucleotide synthesis, to generate the materials needed for the gSynth and Addamer based DNA synthesis methods. Accordingly, disclosed here are compositions and methods that combine gSynth and Addamer technologies with high-throughput oligonucleotide synthesis to generate routine high-fidelity gene (target nucleic acid) synthesis.

### CROSS-REFERENCE TO RELATED APPLICATIONS

This application claims priority to, and the benefit of, U.S. Provisional Application No. 63/186,871, filed May 11, 2021, the contents of which are incorporated herein by reference in their entireties for all purposes.

### SEQUENCE LISTING

The instant application contains a Sequence Listing which has been submitted in ASCII format via EFS-Web and is hereby incorporated by reference in its entirety. Said ASCII copy, created on May 11, 2022, is named “DNWR-010_001 WO_SeqList.txt” and is about 23,462 bytes in size.

### BACKGROUND

There is a need in the art for gene assemblies that efficiently produce arbitrary double stranded DNA sequences with high fidelity and high throughput. Recent advances for gene assembly include double stranded DNA assembly methods referred to as gSynth methods, which are described in detail in PCT Application No. PCT/US2020/051838, published as WO2021055962A1. gSynth methods yield high fidelity DNA sequence assemblies, however there is a need to increase the throughput of gSynth methods, as the use of high-throughput DNA synthesis techniques, such as array-based oligonucleotide synthesis (see Lipshutz, R. J. et al., High density synthetic oligonucleotide arrays. Nature Genetics volume 21, pages20-24, 1999) to produce the required materials for gSynth methods has required intermediate amplification (Saiki R. K. et al., Enzymatic amplification of beta-globin genomic sequences and restriction site analysis for diagnosis of sickle cell anemia. Science 20 Dec. 1985: Vol. 230, Issue 4732, pp. 1350-1354) and processing of the duplex elements.

In addition to gSynth methods, environmentally-friendly (e.g. “green”) double-stranded DNA assembly/synthesis methods have been recently developed that use as a fundamental building double-stranded DNA constructs referred to as “Addamers”. Addamers are double-stranded double-hairpin structures that carry a DNA payload as well as a variety of control elements that allow the manipulation of the sequence. The Addamer elements may include but are not limited to binding sites for restriction endonucleases (REs), binding sites for Type II S restriction endonucleases (IISREs), a payload DNA sequence, and hairpin structures that range from simple GNA motifs (Yoshizawa S et al., GNA Trinucleotide Loop Sequences Producing Extraordinarily Stable DNA Minihairpins. Biochemistry 1997, 36, 16, 4761-4767) to complex three dimensional Aptamers with high affinity ligand binding (Ellington. A. D. and Szostak, J. W. In vitro selection of RNA molecules that bind specific ligands. Nature volume 346, pages818-822. 1990; Tuerk C et al., Systematic evolution of ligands by exponential enrichment: RNA ligands to bacteriophage T4 DNA polymerase. Science 3 Aug. 1990: Vol. 249, Issue 4968, pp. 505-510).

Disclosed herein are compositions and methods that utilize high-throughput oligonucleotide synthesis methods, including array-based oligonucleotide synthesis, to generate the materials needed for the gSynth and Addamer based DNA synthesis methods described above and herein. Accordingly, disclosed here are compositions and methods that combine gSynth and Addamer technologies with high-throughput oligonucleotide synthesis to generate routine high-fidelity gene (target nucleic acid) synthesis.

### SUMMARY

The present disclosure provides compositions comprising two or more pluralities of nucleic acid molecules, wherein each of the pluralities of nucleic acid molecules comprises two or more species of nucleic acid molecules, wherein different species of nucleic acid molecules comprise different nucleic acid sequences, wherein within the two or more pluralities of nucleic acid molecules there is at least one set of corresponding pluralities such that when the at least one set of corresponding pluralities are combined in a single reaction volume, nucleic acid molecules from different pluralities within the set hybridize together to form at least one species of hybridized complex.

In some aspects, the compositions of the present disclosure comprise about: a) six; b) ten; c) 15; d) 20; or e) 50 pluralities of nucleic acid molecules.

In some aspects of the compositions of the present disclosure, each plurality of nucleic acid molecules comprises at least about 25 different species of nucleic acid molecules.

In some aspects of the compositions of the present disclosure, each set of corresponding pluralities comprises the same number of pluralities.

In some aspects of the compositions of the present disclosure, the at least one species hybridized complex comprises one nucleic acid species from each of the pluralities within the set of corresponding pluralities.

In some aspects of the compositions of the present disclosure, the two or more pluralities of nucleic acid molecules are present in separate volumes.

In some aspects of the compositions of the present disclosure, the at least one set of corresponding pluralities comprises at least about: a) two; b) three; c) four; or d) five pluralities of nucleic acid molecules.

In some aspects of the compositions of the present disclosure, the number of sets of corresponding pluralities is equal to: X!/Y! (X−Y)!, wherein X is equal to the total number of pluralities of nucleic acid molecules, and Y is equal to the number of species of nucleic acid that hybridize together to form a single hybridized complex.

In some aspects of the compositions of the present disclosure: a) the at least one set of corresponding pluralities comprises two pluralities, and the at least one hybridized complex comprises two nucleic acid molecules; b) the at least one set of corresponding pluralities comprises three pluralities, and the at least one hybridized complex comprises three nucleic acid molecules; or c) the at least one set of corresponding pluralities comprises four pluralities, and the at least one hybridized complex comprises four nucleic acid molecules.

In some aspects of the compositions of the present disclosure, when a set of corresponding pluralities is combined in a single reaction, at least about five different hybridized complex species are formed.

In some aspects of the compositions of the present disclosure, the different species of nucleic acid molecules within a single plurality of nucleic acid molecules are not complementary to each other.

The present disclosure provides methods of producing at least one Addamer, the method comprising: a) providing a composition of the present disclosure; b) combining at least one set of corresponding pluralities of nucleic acid molecules in a single reaction volume such that at least one species of hybridized complex is formed; c) contacting the at least one species of hybridized complex with at least one ligase enzyme to form that at least one Addamer capped at both ends by hairpins.

In some aspects, the methods of the present disclosure further comprise treating the products of step (c) with an exonuclease, thereby purifying properly ligated Addamers.

In some aspects, the methods of the present disclosure further comprise contacting the at least one species of hybridized complex with a MutS enzyme.

In some aspects, an Addamer comprises: a) a first Type II S restriction endonuclease (IISRE) sequence; b) a payload sequence; c) an at least second IISRE sequence; and at least one end of the Addamer comprises a hairpin structure.

In some aspects, an Addamer comprises a hairpin structure at both ends of the Addamer.

In some aspects, an Addamer comprises: a) a first IISRE sequence; b) a second IISRE sequence; c) a payload sequence; and d) an at least third IISRE sequence.

In some aspects, an Addamer comprises: a) a first IISRE sequence; b) a second IISRE sequence; c) a payload sequence; d) a third IISRE sequence; and e) an at least fourth IISRE sequence.

In some aspects, an Addamer further comprises a multiple cloning site (MCS) sequence, wherein the MCS sequence comprises one or more restriction endonuclease sequences.

In some aspects, at least one of the IISRE sequences are selected from a MlyI sequence, a NgoA VII sequence, SspD5I sequence, an AlwI sequence, a BccI sequence, a BcefI sequence, a PleI sequence, a BceAI sequence, a BceSIV sequence, a BscAI sequence, a BspD6I sequence, a FauI sequence, an EarI sequence, a BspQI sequence, a BfuAI sequence, a PaqCI sequence, an Esp3I sequence, a BbsI sequence, a BbvI sequence, a BtgZI sequence, a FokI sequence, a BsmFI sequence, a BsaI sequence, a BcoDI sequence and a HgaI sequence.

In some aspects, an at least one hairpin structure comprises an aptamer sequence. In some aspects, the aptamer sequence is selected from a pL1 aptamer sequence, a Thrombin 29-mer apatamer sequence, an S2.2 aptamer sequence, an ART1172 aptamer sequence, an R12.45 aptamer sequence, a Rb008 aptamer sequence and a 38NT SELEX apatamer sequence.

The present disclosure provides methods of synthesizing a nucleic acid molecule comprising a target nucleic acid sequence, the method comprising: a) providing the composition of anyone of the preceding claims, wherein the composition comprises a set of corresponding pluralities of nucleic acid molecules such that when the set of corresponding pluralities are combined in a single reaction volume, nucleic acid molecules from different pluralities within the set hybridize together to form two or more species of hybridized complexes, wherein the two or more species of hybridized complexes comprises fragments of the target nucleic acid sequence; b) combining the set of corresponding pluralities of nucleic acid molecules in a single reaction volume such that the two or more species of hybridized complexes are formed; c) contacting the two or more species of hybridized complexes with at least one ligase enzyme to form two or more species of Addamers capped at both ends by hairpins; d) assembling the two or more species of Addamers to synthesize the nucleic acid molecule comprising the target nucleic acid sequence.

In some aspects of the methods of the present disclosure, assembling the two or more species of Addamers comprises treating the Addamers with: a) one or more restriction enzymes; and b) one or more ligases, either concurrently or sequentially, thereby assembling the nucleic acid molecule comprising the target nucleic acid sequence.

In some aspects of the methods of the present disclosure, assembling the two or more species of Addamers comprises treating the Addmers with: a) modified Cas9 exhibiting nickase activity in combination with at least one guide RNA; and b) one or more ligases, either concurrently or sequentially, thereby assembling the nucleic acid molecule comprising the target nucleic acid sequence.

Any of the aspects and/or embodiments described above and herein can be combined with any other aspect and/or embodiment described above and herein.

Unless otherwise defined, all technical and scientific terms used herein have the same meaning as commonly understood by one of ordinary skill in the art to which this disclosure belongs. In the Specification, the singular forms also include the plural unless the context clearly dictates otherwise; as examples, the terms “a,” “an,” and “the” are understood to be singular or plural and the term “or” is understood to be inclusive. By way of example, “an element” means one or more element. Throughout the specification the word “comprising,” or variations such as “comprises” or “comprising,” will be understood to imply the inclusion of a stated element, integer or step, or group of elements, integers or steps, but not the exclusion of any other element, integer or step, or group of elements, integers or steps. About can be understood as within 10%, 9%, 8%, 7%, 6%, 5%, 4%, 3%, 2%, 1%, 0.5%, 0.1%, 0.05%, or 0.01% of the stated value. Throughout the specification, the recitation of ranges of values described as “between x and y” or “between x to y”, wherein x and y are two values, is understood to be inclusive of x and y. Unless otherwise clear from the context, all numerical values provided herein are modified by the term “about.”

Although methods and materials similar or equivalent to those described herein can be used in the practice or testing of the present disclosure, suitable methods and materials are described below. All publications, patent applications, patents, and other references mentioned herein are incorporated by reference in their entirety for all purposes. The references cited herein are not admitted to be prior art to the claimed invention. In the case of conflict, the present Specification, including definitions, will control. In addition, the materials, methods, and examples are illustrative only and are not intended to be limiting. Other features and advantages of the disclosure will be apparent from the following detailed description and claims.

### BRIEF DESCRIPTION OF THE DRAWINGS

The above and further features will be more clearly appreciated from the following detailed description when taken in conjunction with the accompanying drawings.

FIG. 1 shows a schematic depicting generation of Addamers from chemically synthesized oligonucleotide pairs. FIG. 1 depicts constructing a DNA Addamer from two single-stranded DNA molecules. The top and bottom strands, the DUPLEX INSERT and two nick sites are as indicated. The nucleotide sequences of ten or more nucleotides presented in FIG. 1 are put forth in SEQ ID NOs: 1-7

FIG. 2 depicts a two-strand system with pairwise combination of pools to generate Addamer sets for gene assembly. The panel of the left depicts pool locations for the top and bottom strands for Addamer generation. Each combination of 6 pools provides unique combination of complementary Top and Bottom strands. There are 15 possible pairwise combination from 6 pools, this corresponds to the genes A-O. The panel on the right shows the relationship between the number of pools and the number of possible gene assemblies, the number of Addamer gene sets per pool and the total number of different oligonucleotides per pool.

FIG. 3 depicts Addamer generation from multiple oligonucleotide array pools: two strand and three strand designs for Addamer generation. For 300 base strands the overlapping complementary regions are long, 200 base pairs for the two-strand system and 200 base pairs between the left and middle strands and 100 base pairs for the middle and right strands, for the three-strand system. For the two-strand system there are two nicks resulting from successful hybridization and three nicks for the three-strand system. These nicks are rapidly and efficiently resolved by T4 DNA ligase.

FIG. 4 depicts three strand System with three-way combination of pools to generate Addamer sets for gene assembly. The panel of the left depicts pool locations for the Left, Middle and Right strands for Addamer generation. Each combination of 6 pools provides unique combination of complementary Left, Middle and Right strands. There are 20 possible pairwise combination from 6 pools, this corresponds to the genes A-T. The panel on the right shows the relationship between the number of pools and the number of possible gene assemblies, the number of Addamer Gene Sets per pool and the total number of different oligonucleotides per pool.

FIG. 5 depicts three strand system by six pool array layouts. FIG. 5 shows on the left the combinations map for a three-strand system over six oligonucleotide array pools, and on the right an example array layout. Each combination of three pools produces a single gene set of Addamers. Here Gene S (light purple) is constructed from five Addamers encoded by five distinct oligonucleotides (A, B, C, D and E) on each Pool 1, Pool 4 and Pool 6.

FIG. 6. depicts a schematic showing use of a pooled approach, for combining Addamers to generate high fidelity gene sequences. Internal IISRE sites denoted by black speckles. gSynth sites denoted by the numbered squares.

FIG. 7 is an exemplary schematic of an Addamer, which is a double-stranded nucleic acid molecule comprising a hairpin at both ends.

FIG. 8 shows exemplary schematics of various Addamer designs of the present disclosure Show are several examples of Addamer design each Addamer type possesses a payload with flanking IISRE sites, a means of attachment to a solid support and double hairpins to promote exonuclease resistance. Shown in the key are some possible IISRE and RE sites as well as the thrombin aptamer hairpin.

FIG. 9 is an exemplary schematic of a nucleic acid synthesis method of the present disclosure that comprises the use of Addamers of the present disclosure. Initially the 1^{st} and 2^{nd} Addamers are attached using DNA ligation to MCS bearing attachments studs, which have already been loaded onto a solid support. Donor and Acceptor constructs are generated in separate volumes. Donor and Acceptor constructs are treated with distinct IISREs to generate ligatable ends. In this diagram, the Acceptor is generated by digestion with the R1 IISRE, the released end and enzyme are discarded by rinsing. The Donor construct is generated by digestion with the purple L2 enzyme. The Donor construct solution (carrying the L2 enzyme) is transferred to the Acceptor well and ligated using T4 DNA Ligase, which has a high efficiency of >80% for 2, 3 or 4-base sticky end ligation. The well is treated with exonuclease and rinsed. The resulting attached Addamer construct is then ready for subsequent cycles of elongation.

FIG. 10A, FIG. 10B, FIG. 10C, FIG. 10D, FIG. 10E, FIG. 10F, FIG. 10G and FIG. 10H are exemplary schematics of a nucleic acid synthesis method of the present disclosure that comprises the use of Addamers of the present disclosure to synthesize a 27-nucleotide long target nucleic acid molecule. The nucleotide sequence of ten or more nucleotides presented in FIG. 10A corresponds to that put forth in SEQ ID NO: 8. The nucleotide sequences of ten or more nucleotides presented in FIG. 10E correspond to those put forth in SEQ ID NOs: 9-10. The nucleotide sequences of ten or more nucleotides presented in FIG. 10G correspond to those put forth in SEQ ID NOs: 11-22. The nucleotide sequences of ten or more nucleotides presented in FIG. 10H correspond to those put forth in SEQ ID NOs: 23-33.

FIG. 11A-11F is a schematic overview of double-stranded geometric synthesis (gSynth) of the present disclosure.

FIG. 11A is a sequence that is to be synthesized using the double-stranded gSynth methods of the present disclosure. Parts of the sequence that are in bold and underlined correspond to 4-mer overhangs that have been selected, thus defining the fragments that will be used to synthesize the entire sequence. The nucleotide sequences of ten or more nucleotides presented in FIG. 11A corresponds to SEQ ID NO: 34.

FIG. 11B shows the individual double-stranded nucleic acid fragments of the sequence shown in FIG. 11A that will be used in the double-stranded gSynth methods of the present disclosure to construct the sequence shown in FIG. 11A. These fragments are chosen based on the sites selected in FIG. 11A. The nucleotide sequences of ten or more nucleotides presented in FIG. 11B correspond to SEQ ID NOs: 35-62.

FIG. 11C is a schematic of a binary tree that shows the order in which the fragments in FIG. 11B are to be assembled to generate the sequence shown in FIG. 11A.

FIG. 11D is a schematic of the first round of ligations in the double-stranded gSynth method to synthesize the sequence shown in FIG. 11A. In the first ligation round, Fragments 1 and 2, Fragments 3 and 4, Fragments 5 and 6, Fragments 7 and 8, Fragments 9 and 10, Fragments 11 and 12, and Fragments 13 and 14 are hybridized via their complementary 5′ overhangs and then ligated together to create Fragment 1+2, Fragment 3+4, Fragment 5+6, Fragment 7+8, Fragment 9+10, Fragment 11+12, and Fragment 13+14. The nucleotide sequences of ten or more nucleotides presented in FIG. 11D correspond to SEQ ID NOs: 35-62.

FIG. 11E is a schematic of the second round of ligations in the double-stranded gSynth method to synthesize the sequence shown in FIG. 11A. In the second ligation round, Fragments 1+2 and 3+4, Fragments 5+6 and 7+8, and Fragments 11+12 and 13+14 are hybridized via their complementary 5′ overhangs and then ligated together to create Fragment 1+2+3+4, Fragment 5+6+7+8, and Fragment 11+12+13+14. The nucleotide sequences of ten or more nucleotides presented in FIG. 11E correspond to SEQ ID NOs: 63-76.

FIG. 11F is a schematic of the third round of ligations in the double-stranded gSynth method to synthesize the sequence shown in FIG. 11A. In the third ligation round, Fragments 1+2+3+4 and 5+6+7+8, and Fragments 9+10 and 11+12+13+14 are hybridized via their complementary 5′ overhangs and then ligated together to create Fragment 1+2+3+4+5+6+7+8 and Fragment 9+10+11+12+13+14. The nucleotide sequences of ten or more nucleotides presented in FIG. 11F correspond to SEQ ID NOs: 77-84.

FIG. 11G is a schematic of the fourth and final round of ligations in the double-stranded gSynth method to synthesize the sequence shown in FIG. 11A. In the fourth ligation round, Fragments 1+2+3+4+5+6+7+8 and 9+10+11+12+13+14 are hybridized via their complementary 5′ overhangs and ligated together, thereby producing the sequence shown in FIG. 1A. The nucleotide sequences of ten or more nucleotides presented in FIG. 11G correspond to SEQ ID NOs: 85-88.

FIG. 12 shows an exemplary processing and analysis of a target nucleic acid sequence to be synthesized using the methods of the present disclosure. A full-length sequence of 431 bp was divided into five variably sized fragments (F1-F5). These fragments are the payloads of Addamers used to produce the final sequence. A computer program was used to reliably predict well-spaced 4-base overhang sites that are compatible in a ligation reaction, as well as other features, such as optimum GC content. The overall sequence was analysed for the presence of IISRE sites (BsmFI, FokI, BtgZI, SfaNI). Sites present in the target sequence exclude certain IISREs for purposes of assembly.

FIG. 13 shows the Addamers corresponding to the fragments identified in FIG. 12. The payload of the Addamer, which is the predicted fragment (blue arrows indicate 5′->3′ orientation), is flanked with IISRE sites (e.g. BsaI for internal fragments). The 4-base overhangs are shown in light orange boxes (upper strand) and light green boxes (lower strand). For the end fragments F1 and F5, the external IISRE sites are different (BbsI) to allow a subsequent assembly after cloning of the sequence. Fragments F1 and F5 also possess forward and reverse primer sites (Extended T7 and Extended T3) for amplification as well as RE sites (XbaI and EcoRI) for cloning. The nucleotide sequences of ten or more nucleotides presented in FIG. 11G correspond to SEQ ID NOs: 89-108.

FIG. 14 shows the verification of the target nucleic acid sequence assembled using the Addamers presented in FIG. 13 by sequencing. Clones were picked and plasmids were sequenced using capillary sequencing. Reverse and forward sequences aligned perfectly, verifying the accuracy of Addamer based assembly from pools of oligonucleotides.

FIG. 15 shows the design of assembly procedures for additional target nucleic acid sequences assembled using the methods of the present disclosure.

### DETAILED DESCRIPTION

The present disclosure is directed to compositions and methods that utilize pooled nucleic acid molecules to generate double-stranded or partially double-stranded nucleic acid molecules, including, but not limited to, Addamers, for use in double stranded DNA assembly/syntehsis methods, including, but not limited to gSynth-based methods and Addamer-based methods. That is, the compositions and methods described herein improve existing gSynth-based and Addamer-based DNA assembly/synthesis methods by taking advantage of the very high oligonucleotide production possibility of pooled nucleic acid molecules, without the need for intervening amplification of duplexes, which requires large amounts of purified nucleotides and error prone DNA polymerases. In some aspects, these pooled nucleic acid molecules can be produced using array-based oligonucleotide synthesis methods.

In one aspect, a number of oligonucleotide pools can be generated, then combined to allow individual genes to be constructed uniquely from the Addamers generated in those combined pools. For example, using the gSynth algorithm, any gene can be deconstructed into a relatively small number of fragments, with unique high-fidelity overhanging sequences at each junction, allowing straightforward sticky-ended ligation to produce the final product, similar to a Golden Gate assembly approach (Pryor J. M. et al., Enabling one-pot Golden Gate assemblies of unprecedented complexity using data-optimized assembly design. PLOS ONE 15(9): e0238592. 2020). It is important to note that Golden Gate assembly works best with cloned sequences, with uniformly digested, selected high fidelity overlaps. If the duplex sequence is 500 base pairs (assuming two 300 base oligos combined to generate an Addamer with 100 bases for control element, leaves 500 bases or 250 base pairs of duplex sequence) then ligation of five contiguous sequences will produce a 2.5 kb gene. In some aspects, the user of the methods of the present disclosure can widely vary the length of any individual gene fragment, to optimally fit assembly, IISREs site availability and secondary structure parameters.

In some aspects, Addamers (whose structure is described in further detail herein) can be generated by the unique combination of a pair of complementary DNA strands that hybridize to one another forming a double-stranded double hairpin structure with a pair of unresolved nicks. These nicks can be repaired by application of DNA ligase, such as T4 DNA ligase. Once the nicks are resolved, possible mismatches can be removed by His-tagged MutS protein combined with an affinity reagent (Wang J et al., Directly fishing out subtle mutations in genomic DNA with histidine-tagged Thermus thermophilus MutS. Volume 547, Issues 1-2, 22 Mar. 2004, Pages 41-47) In some aspects, all non-Addamer DNA is removed by application of T7 exonuclease.

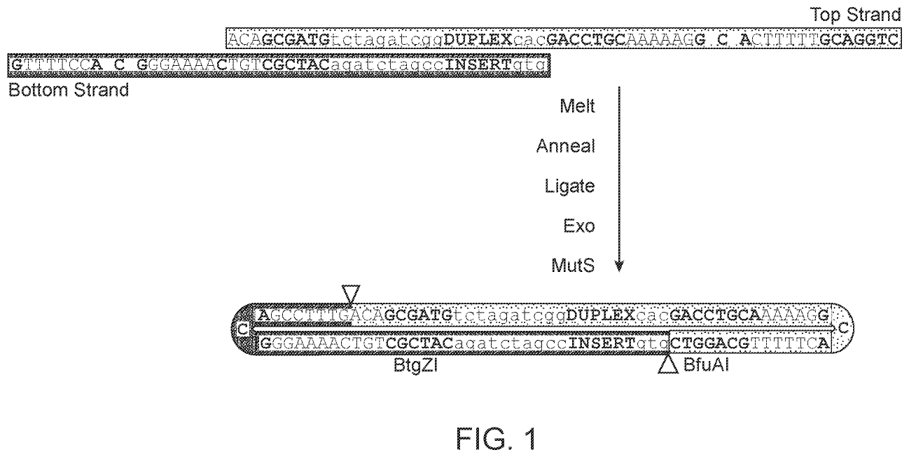

A DNA Addamer can be constructed from two single-stranded DNA molecules. The DUPLEX INSERT can be large, on the order of 250 base pairs (this allows for 100 bases of control sequence from an estimated single-stranded oligonucleotide size of 300 bases). As the total sequence amount can be high, the fidelity of hybridization both in terms of selectivity and duplex recovery will also be high. Annealing can produce a nearly completely double stranded sequence with stable hairpins. The two ‘nicks’ can be efficiently and quickly repaired by a short treatment with T4 DNA ligase. The limited ligation can be combined with the high-fidelity hybridization means that the only T7 exonuclease resistant material can be the desired Addamer product. Additionally, treatment can be done with His-tagged Taq MutS protein, to bind possible mismatched base pairs, followed by contact with Ni-NTA (Nickel nitriloacetic acid) agarose affinity resin, to remove MutS bound mismatch containing DNA, leaving substantially enriched pure desired product (see FIG. 1).

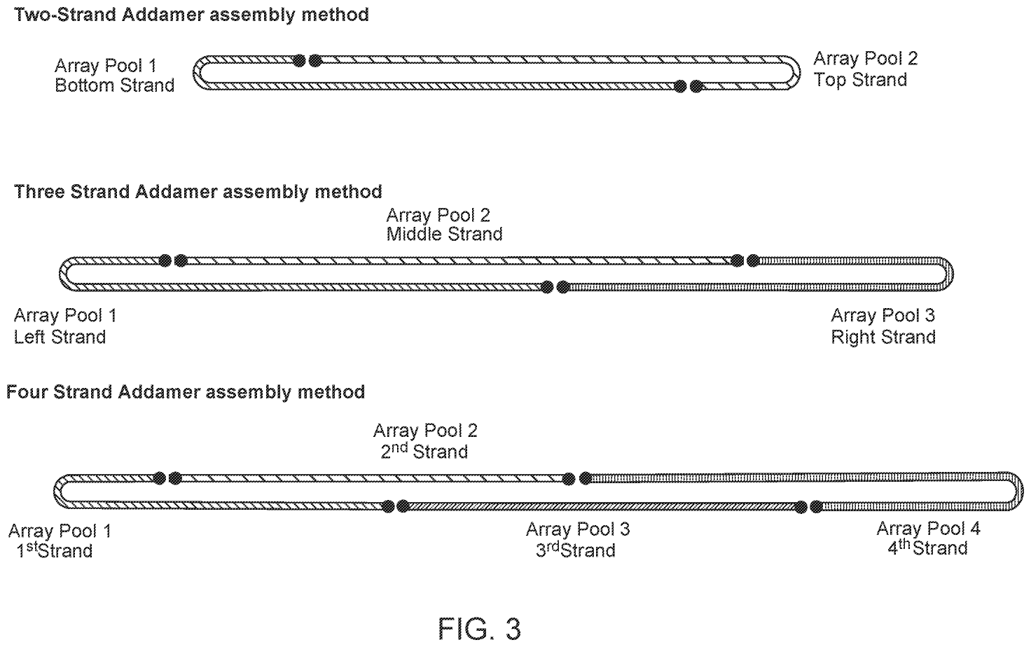

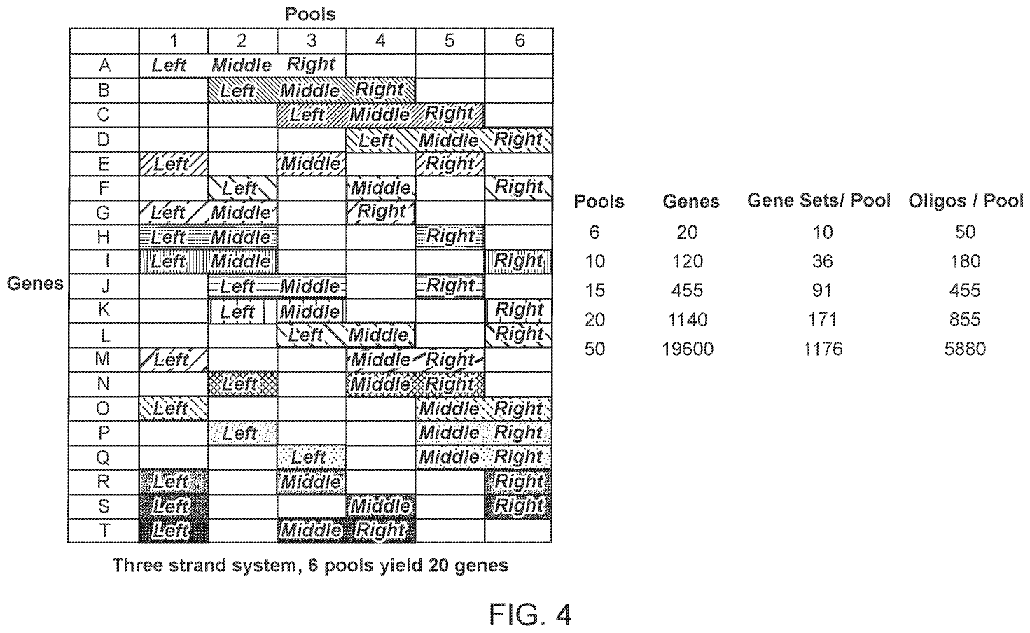

That is, in some aspects an Addamer of the present disclosure can be produced by hybridizing a first single-stranded nucleic acid molecule and a second single-stranded nucleic acid molecule, wherein the first single-stranded nucleic acid molecule comprises a first region that is complementary to a second region on the second single-stranded nucleic acid molecule and a second region that is self-complementary, and the second single-stranded nucleic acid molecule comprises a first region that is self-complementary and a second region that is complementary to a first region on the first single-stranded nucleic acid molecule, as shown in the top panel of FIG. 3. The first single-stranded nucleic acid molecule and the second single-stranded nucleic acid molecule are hybridized together to produce a hybridized complex. The hybridized complex can then be optionally contacted with the enzyme MutS, which binds to mismatched bases and exposes DNA to exonuclease digestion. The hybridized complex can then be contacted with a ligase enzyme to form the double-stranded Addamer structure capped at both ends by hairpins. Following contact with the ligase enzyme, the product can be contacted with T7 exonuclease to purify and enrich for properly formed Addamers. This method is referred to herein as the “two-strand Addamer assembly method”.

Accordingly, the present disclosure provides methods of producing the Addamers described herein, the methods comprising: a) providing a first single-stranded nucleic acid molecule and a second single-stranded nucleic acid molecule, wherein the sequences of the first single-stranded nucleic acid molecule and second single-stranded nucleic acid molecule comprise portions of the Addamer that is to be produced, wherein the first single stranded nucleic acid molecule comprises a first region that is complementary to a second region on the second single-stranded nucleic acid molecule and the second region that is self-complementary, and the second single-stranded nucleic acid molecule comprises a first region that is self-complementary and a second region that is complementary to a first region on the first single-stranded nucleic acid molecule; b) hybridizing the first single-stranded nucleic acid molecule and the second single-stranded nucleic acid molecule; and c) contacting the partially double-stranded nucleic acid molecule with a ligase enzyme to form a double-stranded Addamer structure capped at both ends by hairpins.

In some aspects, the preceding methods can further comprise treating the products of step (c) with an exonuclease, thereby purifying properly ligated Addamers.

In some aspects, the preceding methods can further comprise after step (b) and before step (c), contacting the partially double-stranded nucleic acid molecule with a MutS enzyme

In some aspects, Addamers can be produced by hybridizing a first single-stranded nucleic acid molecule, a second single-stranded nucleic acid molecule and a third single-stranded nucleic acid molecule, wherein the first single-stranded nucleic acid molecule comprises a first region that is complementary to a first region on the second single-stranded nucleic acid molecule and a second region that is self-complementary, the second single-stranded nucleic acid molecule comprises first region that is complementary to the first region of the first single-stranded nucleic acid molecule and a second region that is complementary to the first region of the third single-stranded nucleic acid molecule, and the third single-stranded nucleic acid molecule comprises a first region that is complementary to the second region of the second single-stranded nucleic acid molecule and a second region that is self-complementary, as shown in the middle panel of FIG. 3. In FIG. 3, the first single-stranded nucleic acid molecule is referred to as the left strand, the second single-stranded nucleic acid molecule is referred to as the middle strand, and the third single-stranded nucleic acid molecule is referred to as the right strand. The first single-stranded nucleic acid molecule, the second single-stranded nucleic acid molecule, and the third single-stranded nucleic acid molecule are hybridized together to produce a hybridized complex. The hybridized complex can then be optionally contacted with the enzyme MutS, which binds to mismatched bases and exposes DNA to exonuclease digestion. The hybridized complex can then be contacted with a ligase enzyme to form the double-stranded Addamer structure capped at both ends by hairpins. Following contact with the ligase enzyme, the product can be contacted with T7 exonuclease to purify and enrich for properly formed Addamers. In the three-strand system, the position of the Middle strand relative to the Left and Right strands can be adjusted to balance the degree of hybridization among the strands. There is, however, a trade-off between the amount of self-hybridization to form the hairpins and the amount of cross-hybridization to form the complete Addamer structure. This method is referred to herein as the “three-strand Addamer assembly method”.

Accordingly, the present disclosure provides methods of producing the Addamers described herein, the methods comprising: a) providing a first single-stranded nucleic acid molecule, a second single-stranded nucleic acid molecule and a third single-stranded nucleic acid molecule, wherein the sequences of the first single-stranded nucleic acid molecule, second single-stranded nucleic acid molecule, and third single-stranded nucleic acid molecule comprise portions of the Addamer that is to be produced, wherein the first single-stranded nucleic acid molecule comprises a first region that is complementary to a first region on the second single-stranded nucleic acid molecule and a second region that is self-complementary, the second single-stranded nucleic acid molecule comprises first region that is complementary to the first region of the first single-stranded nucleic acid molecule and a second region that is complementary to the first region of the third single-stranded nucleic acid molecule, and the third single-stranded nucleic acid molecule comprises a first region that is complementary to the second region of the second single-stranded nucleic acid molecule and a second region that is self-complementary; b) hybridizing the first single-stranded nucleic acid molecule, the second single-stranded nucleic acid molecule, and the third single-stranded nucleic acid molecule; and c) contacting the partially double-stranded nucleic acid molecule with a ligase enzyme to form a double-stranded Addamer structure capped at both ends by hairpins.

In some aspects, the preceding methods can further comprise treating the products of step (c) with an exonuclease, thereby purifying properly ligated Addamers.

In some aspects, the preceding methods can further comprise after step (b) and before step (c), contacting the partially double-stranded nucleic acid molecule with a MutS enzyme.

In some aspects, Addamers can be produced by hybridizing a first single-stranded nucleic acid molecule, a second single-stranded nucleic acid molecule, a third single-stranded nucleic acid molecule, and a fourth single wherein the first single-stranded nucleic acid molecule comprises a first region that is complementary to a first region on the second single-stranded nucleic acid molecule and a second region that is self-complementary, the second single-stranded nucleic acid molecule comprises first region that is complementary to the first region of the first single-stranded nucleic acid molecule and a second region that is complementary to the first region of the third single-stranded nucleic acid molecule, the third single-stranded nucleic acid molecule comprises a first region that is complementary to the second region of the second single-stranded nucleic acid molecule and second region that is complementary to a first region on the fourth single-stranded nucleic acid molecule, and the fourth single-stranded nucleic acid molecule comprises a first region that is complementary to the second region of the third-single-stranded nucleic acid molecule and a second region that is self-complementary, as shown in the bottom panel of FIG. 3. The first single-stranded nucleic acid molecule, the second single-stranded nucleic acid molecule, the third single-stranded nucleic acid molecule, and the fourth single-stranded nucleic acid molecules are hybridized together to produce a hybridized complex. The hybridized complex can then be optionally contacted with the enzyme MutS, which binds to mismatched bases and exposes DNA to exonuclease digestion. The hybridized complex can then be contacted with a ligase enzyme to form the double-stranded Addamer structure capped at both ends by hairpins. Following contact with the ligase enzyme, the product can be contacted with T7 exonuclease to purify and enrich for properly formed Addamers. In the three-strand system, the position of the Middle strand relative to the Left and Right strands can be adjusted to balance the degree of hybridization among the strands. There is, however, a trade-off between the amount of self-hybridization to form the hairpins and the amount of cross-hybridization to form the complete Addamer structure. This method is referred to herein as the “four-strand Addamer assembly method”.

Accordingly, the present disclosure provides methods of producing the Addamers described herein, the methods comprising: a) providing a first single-stranded nucleic acid molecule, a second single-stranded nucleic acid molecule, a third single-stranded nucleic acid molecule, and a fourth single-stranded nucleic acid molecule, wherein the sequences of the first single-stranded nucleic acid molecule, the second single-stranded nucleic acid molecule, the third single-stranded nucleic acid molecule, and the fourth single-stranded nucleic acid molecule comprise portions of the Addamer that is to be produced, wherein the first single-stranded nucleic acid molecule comprises a first region that is complementary to a first region on the second single-stranded nucleic acid molecule and a second region that is self-complementary, the second single-stranded nucleic acid molecule comprises first region that is complementary to the first region of the first single-stranded nucleic acid molecule and a second region that is complementary to the first region of the third single-stranded nucleic acid molecule, the third single-stranded nucleic acid molecule comprises a first region that is complementary to the second region of the second single-stranded nucleic acid molecule and second region that is complementary to a first region on the fourth single-stranded nucleic acid molecule, and the fourth single-stranded nucleic acid molecule comprises a first region that is complementary to the second region of the third-single-stranded nucleic acid molecule and a second region that is self-complementary; b) hybridizing the first single-stranded nucleic acid molecule, the second single-stranded nucleic acid molecule, and the third single-stranded nucleic acid molecule; and c) contacting the partially double-stranded nucleic acid molecule with a ligase enzyme to form a double-stranded Addamer structure capped at both ends by hairpins.

In some aspects, the preceding methods can further comprise treating the products of step (c) with an exonuclease, thereby purifying properly ligated Addamers.

In some aspects, the preceding methods can further comprise after step (b) and before step (c), contacting the partially double-stranded nucleic acid molecule with a MutS enzyme.

In the context of two-strand Addamer assembly methods, three-strand Addamer assembly methods and/or four-strand Addamer assembly methods, the individual single-stranded nucleic acid molecules that are used for each method can be individually provided in separate pluralities (also referred to herein as “pools”) of nucleic acid molecules, wherein the separate pluralities of nucleic acid molecules are produced using methods such as array-based oligonucleotide synthesis. As would be appreciated by the skilled artisan, array-based oligonucleotide synthesis methods include, but are not limited to, electrochemical methods, light-based chemistry methods, inkjet printing methods or any combination thereof.

That is, gene fragment sets can be distributed over several oligonucleotide pools such that unique pairs of pools would be combined to generate Addamers representing each of the fragment of the final gene (target nucleic acid) construct to be synthesized. As an example, for six oligonucleotide array pools, where each gene is constructed of five fragments, there would be a total of 15 gene constructions possible and each pool would contain the top or bottom five fragments for five different genes. Thus 6 pools of 25 oligonucleotides each, where the oligonucleotides are ˜300 bases long would be sufficient to construct 15 genes of 2.5 kb length.

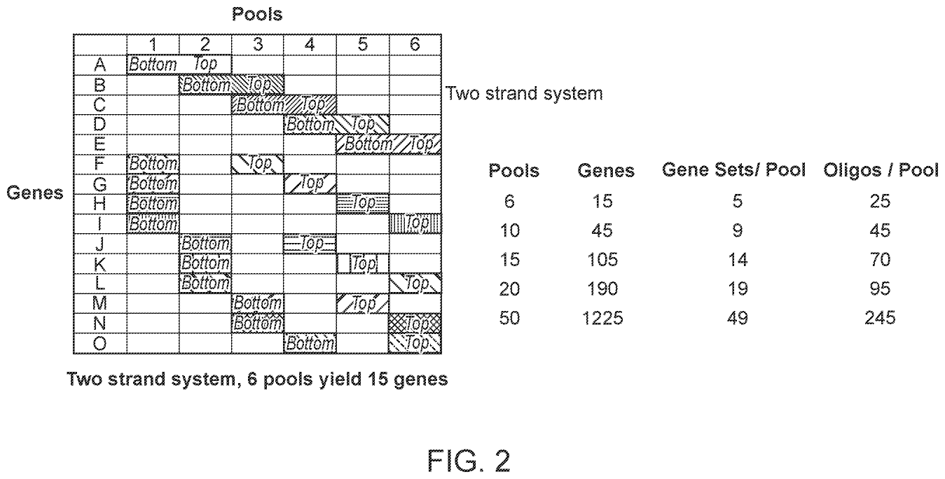

With pairwise combination it is clear that the capacity of current array pools is significantly under-utilized. Even with 50 pools, where 1225 genes could be assembled, the total number of distinct oligonucleotides per pool is only 245 (see FIG. 2). While, with smaller numbers of distinct oligonucleotides per pool, the amount of each specific oligonucleotide is increased, there will be a limit to the total amount of each oligonucleotide needed to effectively assemble a given gene. By increasing the number of strands used to generate Addamers the total number of genes and distinct oligonucleotides per pool is dramatically increased, especially with high numbers of pools.

In another aspect, the three-strand Addamer assembly method can be employed (see FIG. 3), rather than two. With a three-strand system, the number of possible combinations of pool is dramatically increased. For both two and three-strand designs there is a strong selective advantage for formation of specific Addamers, rather than random combinations. Firstly, with using long oligos from array pools, for example 300 bases per strand the overlapping complementary regions is very long, which ensures robust hybridization under normal conditions. Secondly, because the process includes a T7 Exonuclease treatment, to remove non-Addamer DNA, the ligation of the nicks becomes a selective event. Thus, if the hybridization is not correct, the Addamer will not ligate together to form a double hairpin structure and will be degraded by T7 Exonuclease.

In another preferred embodiment a four-strand system can be employed. With four strands and four pools per gene, the number of genes is increased for pool numbers of 10 and above. For example, with 15 pools, a total of 1365 gene can be produced (see Table 1). The increased complexity of the pools provides a more efficient utilization of the oligonucleotide arrays.

TABLE 1

| Pools | Genes | Gene Sets/Pool | Oligos/Pool |
|---|---|---|---|
| 6 | 15 | 10 | 50 |
| 10 | 210 | 84 | 420 |
| 15 | 1365 | 364 | 1820 |
| 20 | 4845 | 969 | 4845 |
| 50 | 230300 | 18424 | 92120 |

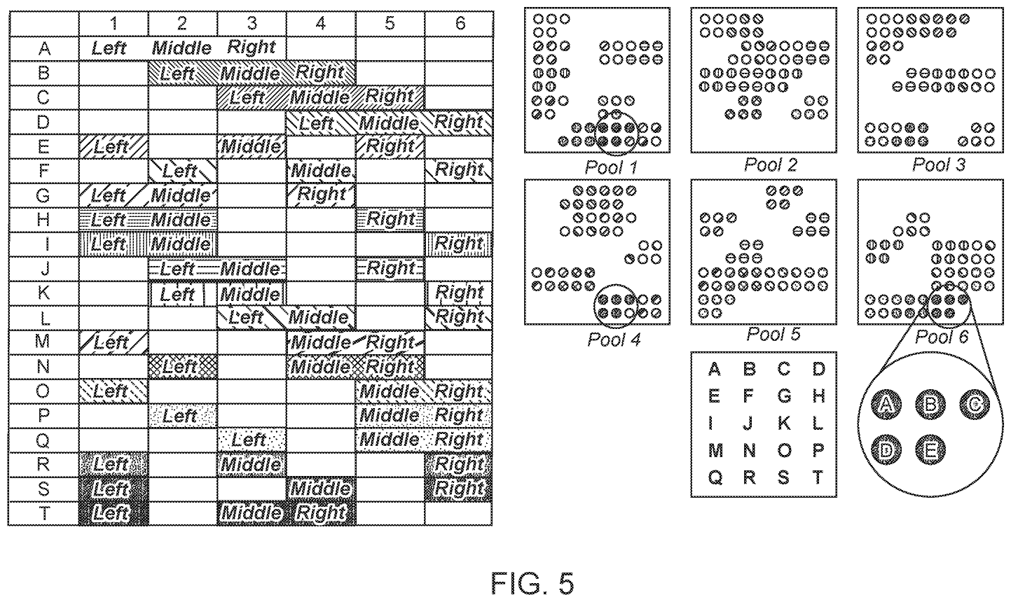

Some commercial oligonucleotide array pools may be generated with an arbitrary number of distinct oligonucleotides. In this case, for a fixed array surface, a smaller number of distinct oligonucleotides will yield a greater total mass for each oligonucleotide sequence. Considering a three-strand system with six pools, each pool will have 50 different oligonucleotide species, thus in combined three pools there will be 150 distinct oligonucleotides to discriminate between (see FIG. 5). Combined pools can be used to generate an arbitrary number of Addamers, but to avoid inappropriate ligation of the overhangs, it is best to keep the number of Addamers in an assembly to a minimum.

Accordingly, the present disclosure provides a composition comprising two or more pluralities of nucleic acid molecules, wherein each of the pluralities of nucleic acid molecules comprises two or more species of nucleic acid molecules, wherein different species of nucleic acid molecules comprise different nucleic acid sequences, wherein within the two or more pluralities of nucleic acid molecules there is at least one set of corresponding pluralities such that when the set of corresponding pluralities are combined in a single reaction volume at least one species of nucleic acid from at least one of the pluralities in the set hybridizes to at least one species of nucleic acid molecule from at least one of the other pluralities in the set to form at least one species of hybridized complex.

Accordingly, the present disclosure provides a composition comprising two or more pluralities of nucleic acid molecules, wherein each of the pluralities of nucleic acid molecules comprises two or more species of nucleic acid molecules, wherein different species of nucleic acid molecules comprise different nucleic acid sequences, wherein within the two or more pluralities of nucleic acid molecules there is at least one set of corresponding pluralities such that when the set of corresponding pluralities are combined in a single reaction volume, nucleic acid molecules from different pluralities within the set hybridize together to form at least one species of hybridized complex.

Accordingly, the present disclosure provides a composition comprising two or more pluralities of nucleic acid molecules, wherein each of the pluralities of nucleic acid molecules comprises two or more species of nucleic acid molecules, wherein different species of nucleic acid molecules comprise different nucleic acid sequences, wherein within the two or more pluralities of nucleic acid molecules there is at least one set of corresponding pluralities such that when the set of corresponding pluralities are combined in a single reaction volume, nucleic acid molecules from each different plurality within the set hybridize together to form at least one species of hybridized complex.

In some aspects of the preceding compositions, a hybridized complex can be any of the hybridized complexes shown in FIG. 3.

In some aspects of the preceding compositions, within each plurality of nucleic acid molecules, a single species of nucleic acid molecule is present in a plurality (i.e. there is more than one copy of that species of nucleic acid molecule present within the plurality).

In some aspects, the preceding compositions can comprise at least about one, or at least about two, or at least about three, or at least about four, or at least about five, or at least about six, or at least about seven, or at least about eight, or at least about nine, or at least about 10, or at least about 11, or at least about 12, or at least about 13, or at least about 14, or at least about 15, or at least about 16, or at least about 17, or at least about 18, or at least about 19, or at least about 20, or at least about 25, or at least about 30, or at least about 35, or at least about 40, or at least about 45, or at least about 50, or at least about 55, or at least about 60, or at least about 65, or at least about 70, or at least about 75, or at least about 80, or at least about 85, or at least about 90, or at least about 95, or at least about 100, or at least about 150, or at least about 200, or at least about 250, or at least about 300, or at least about 350, or at least about 400, or at least about 450, or at least about 500, or at least about 550, or at least about 600, or at least about 650, or at least about 700, or at least about 750, or at least about 800, or at least about 850, or at least about 900, or at least about 950, or at least about 1000, or at least about 1500, or at least about 2000, or at least about 2500, or at least about 3000, or at least about 3500, or at least about 4000, or at least about 4500, or at least about 5000, or at least about 5500, or at least about 6000, or at least about 6500, or at least about 7000, or at least about 7500, or at least 8000, or at least about 8500, or at least about 9000, or at least about 9500, or at least about 10000 pluralities of nucleic acid molecules.

In some aspects, the preceding compositions can comprise can comprise about one, or about two, or about three, or about four, or about five, or about six, or about seven, or about eight, or about nine, or about 10, or about 11, or about 12, or about 13, or about 14, or about 15, or about 16, or about 17, or about 18, or about 19, or about 20, or about 25, or about 30, or about 35, or about 40, or about 45, or about 50, or about 55, or about 60, or about 65, or about 70, or about 75, or about 80, or about 85, or about 90, or about 95, or about 100, or about 150, or about 200, or about 250, or about 300, or about 350, or about 400, or about 450, or about 500, or about 550, or about 600, or about 650, or about 700, or about 750, or about 800, or about 850, or about 900, or about 950, or about 1000, or about 1500, or about 2000, or about 2500, or about 3000, or about 3500, or about 4000, or about 4500, or about 5000, or about 5500, or about 6000, or about 6500, or about 7000, or about 7500, or 8000, or about 8500, or about 9000, or about 9500, or about 10000 pluralities of nucleic acid molecules.

In some aspects of the preceding compositions, each plurality of nucleic acid molecules can comprise at least about one, or at least about two, or at least about three, or at least about four, or at least about five, or at least about six, or at least about seven, or at least about eight, or at least about nine, or at least about 10, or at least about 11, or at least about 12, or at least about 13, or at least about 14, or at least about 15, or at least about 16, or at least about 17, or at least about 18, or at least about 19, or at least about 20, or at least about 25, or at least about 30, or at least about 35, or at least about 40, or at least about 45, or at least about 50, or at least about 55, or at least about 60, or at least about 65, or at least about 70, or at least about 75, or at least about 80, or at least about 85, or at least about 90, or at least about 95, or at least about 100, or at least about 150, or at least about 200, or at least about 250, or at least about 300, or at least about 350, or at least about 400, or at least about 450, or at least about 500, or at least about 550, or at least about 600, or at least about 650, or at least about 700, or at least about 750, or at least about 800, or at least about 850, or at least about 900, or at least about 950, or at least about 1000, or at least about 1500, or at least about 2000, or at least about 2500, or at least about 3000, or at least about 3500, or at least about 4000, or at least about 4500, or at least about 5000, or at least about 5500, or at least about 6000, or at least about 6500, or at least about 7000, or at least about 7500, or at least 8000, or at least about 8500, or at least about 9000, or at least about 9500, or at least about 10000, or at least about 20000, or at least about 30000, or at least about 40000, or at least about 50000, or at least about 60000, or at least about 70000, or at least about 80000, or at least about 90000, or at least about 100000 different species of nucleic acid molecules.

In some aspects of the preceding compositions, each plurality of nucleic acid molecules can comprise about one, or about two, or about three, or about four, or about five, or about six, or about seven, or about eight, or about nine, or about 10, or about 11, or about 12, or about 13, or about 14, or about 15, or about 16, or about 17, or about 18, or about 19, or about 20, or about 25, or about 30, or about 35, or about 40, or about 45, or about 50, or about 55, or about 60, or about 65, or about 70, or about 75, or about 80, or about 85, or about 90, or about 95, or about 100, or about 150, or about 200, or about 250, or about 300, or about 350, or about 400, or about 450, or about 500, or about 550, or about 600, or about 650, or about 700, or about 750, or about 800, or about 850, or about 900, or about 950, or about 1000, or about 1500, or about 2000, or about 2500, or about 3000, or about 3500, or about 4000, or about 4500, or about 5000, or about 5500, or about 6000, or about 6500, or about 7000, or about 7500, or 8000, or about 8500, or about 9000, or about 9500, or about 10000, or about 20000, or about 30000, or about 40000, or about 50000, or about 60000, or about 70000, or about 80000, or about 90000, or about 100000 different species of nucleic acid molecules.

In some aspects of the preceding compositions, the different species of nucleic acid molecules within a single plurality of nucleic acid molecules are not complementary to each other.

In some aspects of the preceding composition, each set of corresponding pluralities comprises the same number of pluralities. In some aspects, the at least one species of hybridized complex comprises one nucleic acid species from each of the pluralities within the set of corresponding pluralities.

In some aspects of the preceding composition, the two or more pluralities of nucleic acid molecules are present in separate volumes (i.e. they are physically separate from each other such as in different containers or on physically distinct parts of an array).

In some aspects of the preceding compositions, a set of corresponding pluralities can comprise at least about two pluralities, at least about three pluralities, at least about four pluralities, at least about five pluralities, at least about six pluralities, at least about seven pluralities, at least about eight pluralities, at least about nine pluralities, or at least about ten pluralities.

In some aspects of the preceding compositions, a set of corresponding pluralities can comprise about two pluralities, about three pluralities, about four pluralities, about five pluralities, about six pluralities, seven pluralities, about eight pluralities, about nine pluralities, or about ten pluralities.

In some aspects of the preceding compositions, a set of corresponding pluralities can comprise two pluralities, three pluralities, four pluralities, five pluralities, six pluralities, seven pluralities, eight pluralities, nine pluralities, or ten pluralities.

In some aspects of the preceding compositions, the number of sets of corresponding pluralities can be equal to:

$$X ! / Y ! ( X - Y ) !$$

wherein X is equal to the total number of pluralities of nucleic acid molecules, and Y is equal to the number of species of nucleic acid that hybridize together to form a single hybridized complex. For example, a composition comprising 10 pluralities of nucleic acid, wherein each of the hybridized complexes are comprised of three species of nucleic acid molecules, would have 120 sets of corresponding pluralities.

In a non-limiting example, if a set of corresponding pluralities comprises two pluralities, then the hybridized complexes will comprise two nucleic acid molecules, one from each of the two pluralities. In a non-limiting example, if a set of corresponding pluralities comprises three pluralities, then the hybridized complexes will comprise three nucleic acid molecules, one from each of the three pluralities. In a non-limiting example, if a set of corresponding pluralities comprises four pluralities, then the hybridized complexes will comprise four nucleic acid molecules, one from each of the four pluralities.

In some aspects of the preceding compositions, when a set of corresponding pluralities is combined in a single reaction, at least about one, or at least about two, or at least about three, or at least about four, or at least about five, or at least about six, or at least about seven, or at least about eight, or at least about nine, or at least about 10, or at least about 11, or at least about 12, or at least about 13, or at least about 14, or at least about 15, or at least about 16, or at least about 17, or at least about 18, or at least about 19, or at least about 20, or at least about 25, or at least about 30, or at least about 35, or at least about 40, or at least about 45, or at least about 50, or at least about 55, or at least about 60, or at least about 65, or at least about 70, or at least about 75, or at least about 80, or at least about 85, or at least about 90, or at least about 95, or at least about 100 different hybridized complex species can be formed.

In some aspects of the preceding compositions, when a set of corresponding pluralities is combined in a single reaction, about one, or about two, or about three, or about four, or about five, or about six, or about seven, or about eight, or about nine, or about 10, or about 11, or about 12, or about 13, or about 14, or about 15, or about 16, or about 17, or about 18, or about 19, or about 20, or about 25, or about 30, or about 35, or about 40, or about 45, or about 50, or about 55, or about 60, or about 65, or about 70, or about 75, or about 80, or about 85, or about 90, or about 95, or about 100 different hybridized complex species can be formed.

In some aspects of the preceding compositions, that least one species of hybridized complex that is formed corresponds to an Addamer of the present disclosure such that if the at least one species of hybridized complex is contacted with a suitable ligase and optionally MutS enzyme, then an Addamer of the present disclosure will be formed from the hybridized complex.

Accordingly, the present disclosure provides a method of producing at least one Addamer of the present disclosure, the method comprising: a) providing a composition of the present disclosure; b) combining at least one set of corresponding pluralities of nucleic acid molecules in a single reaction volume such that at least one species of hybridized complex is formed; c) contacting the at least one species of hybridized complex with a ligase enzyme to form a double-stranded Addamer structure capped at both ends by hairpins. In some aspects, the at least one species of hybridized complex comprises two single-stranded nucleic acid molecules, as put forth in the two-strand Addamer assembly method described herein. In some aspects, the at least one species of hybridized complex comprises three single-stranded nucleic acid molecules, as put forth in the three-strand Addamer assembly method described herein. In some aspects, the at least one species of hybridized complex comprises four single-stranded nucleic acid molecules, as put forth in the four-strand Addamer assembly method described herein.

In some aspects, the preceding methods can further comprise treating the products of step (c) with an exonuclease, thereby purifying properly ligated Addamers.

In some aspects, the preceding methods can further comprise after step (b) and before step (c), contacting the partially double-stranded nucleic acid molecule with a MutS enzyme.

Additionally, the present disclosure provides a method of producing at least one double-stranded fragment of the present disclosure, the method comprising: a) providing a composition of the present disclosure; b) combining at least one set of corresponding pluralities of nucleic acid molecules in a single reaction volume such that at least one species of hybridized complex is formed, wherein the at least one species of hybridized complex comprises the at least one double-stranded fragment. In some aspects, the at least one double-stranded fragment is a fragment for use in a gSynth synthesis method, as described herein.

### Addamer-Based Methods and Compositions of the Present Disclosure

### Addamers

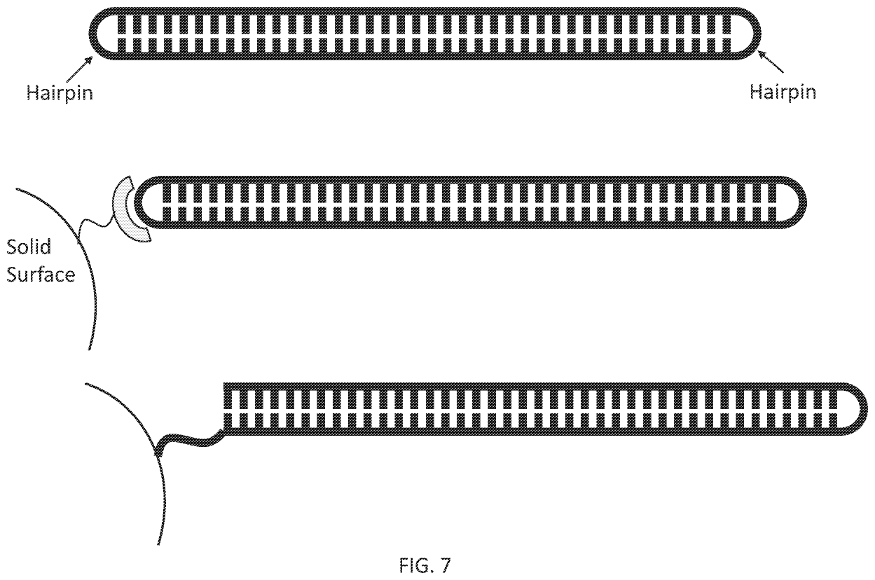

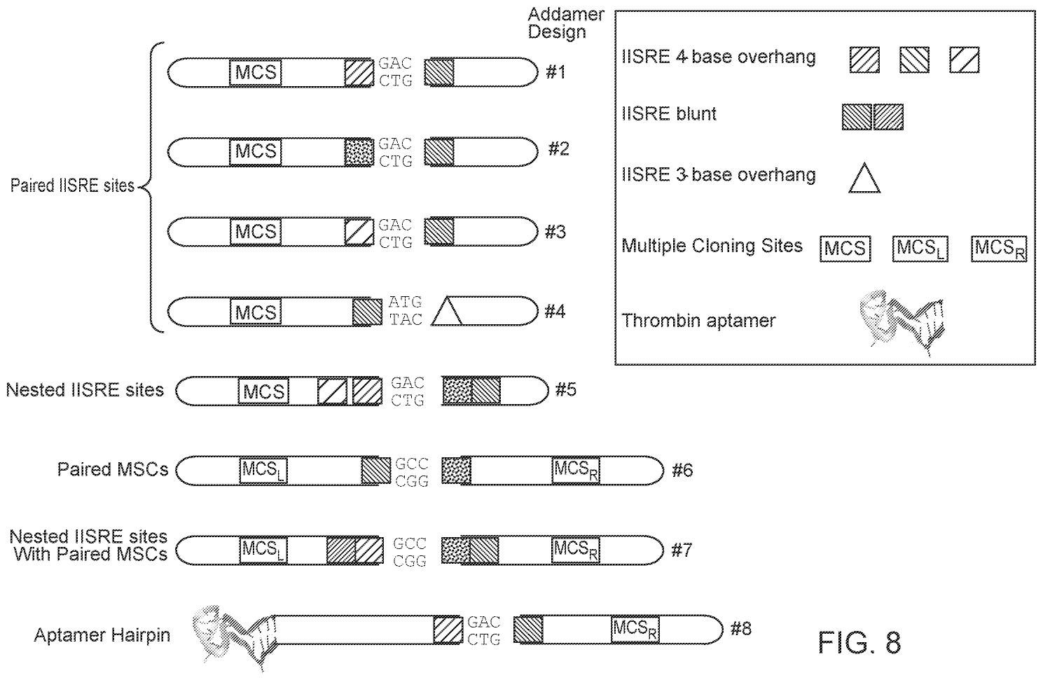

The present disclosure provides a composition comprising at least one Addamer. As used herein, the term Addamer is used to describe a double-stranded nucleic acid molecule comprising a hairpin structure at both ends. In some aspects wherein the Addamer is immobilized to a solid surface, an Addamer may comprise a single hairpin located at the end of the molecule that is not attached to the solid surface. An exemplary schematic of an Addamer and two Addamers immobilized to a solid surface are shown in FIG. 7. An Addamer can comprise one or more features described herein.

In some aspects, an Addamer can comprise, consists essentially of, or consist of DNA.

In some aspects, an Addamer can comprise one or more multiple cloning site (MCS) sequences. In some aspects, an MCS sequence can comprise one or more restriction endonuclease (RE) sequences that can be cleaved with the corresponding restriction endonuclease to generate a 3′ overhang, a 5′ overhang, or a blunt end.

As would be appreciated by the skilled artisan, a “blunt end” is used to describe the end of a DNA fragment in which there are no unpaired nucleotides.

As would be appreciated by the skilled artisan, the term 5′ overhang is used to refer to a single-stranded portion of a partially double-stranded nucleic acid molecule that is located at the 5′ terminus of one of the strands.

As would be appreciated by the skilled artisan, the term 3′ overhang is used to refer to a single-stranded portion of a partially double-stranded nucleic acid molecule that is located at the 3′ terminus of one of the strands.

In some aspects, an Addamer can comprise at least one offset cutting Type II S restriction endonuclease (IISRE) sequences (hereafter “IISRE sequence”) that can be cleaved with a corresponding Type II S restriction endonuclease (hereafter “IISRE”). In some aspects, an Addamer can comprise at least one IISRE sequences. In some aspects, an Addamer can comprise at least three IISRE sequences. In some aspects, an Addamer can comprise at least four IISRE sequences.

In some aspects, the IISRE sequence is a sequence such that cleavage with the corresponding IISRE results in the creation of a “blunt end”.

In some aspects, the IISRE sequence is a sequence such that cleavage with the corresponding IISRE results in the creation of a 5′ overhang that is 1 nucleotide in length. In some aspects, the IISRE sequence is a sequence such that cleavage with the corresponding IISRE results in the creation of a 5′ overhang that is 2 nucleotides in length. In some aspects, the IISRE sequence is a sequence such that cleavage with the corresponding IISRE results in the creation of a 5′ overhang that is 3 nucleotides in length. In some aspects, the IISRE sequence is a sequence such that cleavage with the corresponding IISRE results in the creation of a 5′ overhang that is 4 nucleotides in length. In some aspects, the IISRE sequence is a sequence such that cleavage with the corresponding IISRE results in the creation of a 5′ overhang that is 5 nucleotides in length. In some aspects, the IISRE sequence is a sequence such that cleavage with the corresponding IISRE results in the creation of a 5′ overhang that is about 1 nucleotide to about 5 nucleotides in length.

Non-limiting examples of IISRE sequences, along with their corresponding IISRE and a description of the overhang/blunt end that is created by cleavage of the IISRE sequence and the corresponding IISRE are shown in Table 2. Accordingly, an Addamer can comprise one or more of the IISRE sequences put forth in Table 2.

TABLE 2
Exemplary IISRE sequences and corresponding IISRE

| | | Overhang/Blunt |
|---|---|---|
| IISRE Name | IISRE sequence | end generated |
| MlyI | GAGTC (5/5) | Blunt end |
| NgoAVII | GCCGC (7/7) | Blunt end |
| SspD5I | GGTGA (8/8) | Blunt end |
| AlwI | GGATC (4/5) | 1 nt 5′ overhang |
| BccI | CCATC (4/5) | 1 nt 5′ overhang |
| BcefI | ACGGC (12/13) | 1 nt 5′ overhang |
| PleI | GAGTC (4/5) | 1 nt 5′ overhang |
| BceAI | ACGGC (12/14) | 2 nt 5′ overhang |
| BceSIV | GCTGC (9/11) | 2 nt 5′ overhang |
| BscAI | GCATC (4/6) | 2 nt 5′ overhang |
| BspD6I | GAGTC (4/6) | 2 nt 5′ overhang |
| FauI | CCCGC (4/6) | 2 nt 5′ overhang |
| EarI | CTCTTC (1/4) | 3 nt 5′ overhang |
| BspQI | GCTCTTC (1/4) | 3 nt 5′ overhang |
| BfuAI | ACCTGC (4/8) | 4 nt 5′ overhang |
| PaqCI | CACCTGC (4/8) | 4 nt 5′ overhang |
| Esp3I | CGTCTC (1/5) | 4 nt 5′ overhang |
| BbsI | GAAGAC (2/6) | 4 nt 5′ overhang |
| BbvI | GCAGC (8/12) | 4 nt 5′ overhang |
| BtgZI | GCGATG (10/14) | 4 nt 5′ overhang |
| FokI | GGATG (9/13) | 4 nt 5′ overhang |
| BsmFI | GGGAC (10/14) | 4 nt 5′ overhang |
| BsaI | GGTCTC (1/5) | 4 nt 5′ overhang |
| BcoDI | GTCTC (1/5) | 4 nt 5′ overhang |
| HgaI | GACGC (5/10) | 5 nt 5′ overhang |

In some aspects, a hairpin structure, or hairpin (used interchangeably), located at the end of an Addamer can comprise at least about 1, or at least about two, or at least about three, or at least about four, or at least about five, or at least about six, or at least about seven, or at least about eight, or at least about nine, or at least about ten, or at least about 11, or at least about 12, or at least about 13, or at least about 14, or at least about 15, or at least about 16, or at least about 17, or at least about 18, or at least about 19, or at least about 20, or at least about 21, or at least about 22, or at least about 23, or at least about 24, or at least about 25, or at least about 26, or at least about 27, or at least about 28, or at least about 29, or at least about 30, or at least about 31, or at least about 32, or at least about 33, or at least about 34, or at least about 35, or at least about 36, or at least about 37, or at least about 38, or at least about 39, or at least about 40, or at least about 41, or at least about 42, or at least about 43, or at least about 44, or at least about 45, or at least about 46, or at least about 47, or at least about 48, or at least about 49, or at least about 50 nucleotides.

As described above, Addamers are capped at either end by hairpin structures. The hairpin structures serve several roles. First, the hairpins provide protection against exonuclease digestion for the Addamer. This allows clearance of unreacted intermediates from a given reaction in the methods of the present disclosure, which provides purity, both of Addamers during generation and of products after Addamer elongation. Secondly, the hairpin structures provide means for attachment of Addamers to solid supports. These attachments are generated directly by binding of aptamers, which bind to specific solid-support bound ligands; by ligation through MCS after digestion with conventional REs, such as BamHI; by ligation through lambda phage cos sites; or by hybridization to single stranded solid-support bound anchor NA. Finally, the hairpins described herein allow Addamers to be attached to a solid support (e.g. a bead) without the need for non-natural modifications such as biotin. Thus, the Addamer of the present disclosure can be synthesized using completely natural means, removing the need for small- and or large-scale phosphoramidite synthesis. Accordingly, the Addamers and methods of the present disclosure can allow for faster and less expensive synthesis of nucleic acid molecules and produce less toxic waste.

In some aspects, a hairpin located at the end of an Addamer can comprise a structural sequence that allows for the affinity purification of the Addamer and/or attachment of the Addamer to a solid support (e.g. a bead).

In some aspects, a hairpin located at the end of an Addamer can comprise an enzymatic sequence (e.g. a DNAzyme sequence) that allows for controlled autocleavage.

In some aspects, a hairpin located at the end of an Addamer can comprise one or more restriction enzyme sites. Without wishing to be bound by theory, the one or more restriction enzyme sites in a hairpin can be cleaved with the corresponding restriction enzyme(s) to generate at least one single-stranded overhang, which can subsequently be used to hybridize and/or ligate the cleaved Addamer to a solid support (e.g. a bead) comprising a nucleic acid that is complementary to the at least one single-stranded overhang.

In some aspects, a hairpin located at the end of an Addamer can comprise an aptamer sequence. Without wishing to be bound by theory, an aptamer sequence can be used for affinity purification and/or attachment to a solid support (e.g. a bead). Non-limiting examples of aptamer sequences are shown in Table 3.

TABLE 3
Exemplary Aptamers

| Aptamer | | |
|---|---|---|
| Name | Ligand | Sequence |
| pL1 | Anti-PvLDH | TCGATTGGATTGTGCCGGAAGTGCTGGCTCGA |
| | | (SEQ ID NO: 109) |
| Thrombin | Anti- | AGTCCGTGGTAGGGCAGGTTGGGGTGACT (SEQ |
| 29-mer | thrombin | ID NO: 110) |
| S2.2 | Anti-Muc1 | CAGTTGATCCTTTGGATACCCTG (SEQ ID NO: 111) |
| ART1172 | Anti-VWF | GGCGTGCAGTGCCTTCGGCCGTGCGGTGCCTCCG |
| | | TCACGCCT (SEQ ID NO: 112) |
| R12.45 | Anti- | ACCGTCTGAGCGATTCGTACTTTATTCGGGAGGT |
| | Atrazine | ATCAGCGGG (SEQ ID NO: 113) |
| Rb008 | Anti-ATX | CCTGGACGGAACCAGAATACTTTTGGTCTCCAGG |
| | | (SEQ ID NO: 114) |
| 38NT | Anti- | AAATACCCCCCCTTCGGTGCAAAGCACCGAAGG |
| SELEX | HIV_RT | GGGGGTATTT (SEQ ID NO: 115) |

In some aspects, an Addamer can comprise a lambda phage cos site.

In some aspects, an Addamer can comprise an “N-mer sequence” that comprises a fragment of a nucleic acid that is to be synthesized using one of the methods described herein. The terms “N-mer sequence”, “payload”, “payload sequence”, “N-mer payload” and “duplex insert” are used herein interchangeably.

In some aspects, an N-mer sequence can be about 3 nucleotides in length. In some aspects, an N-mer sequence is 3 nucleotides in length. An N-mer sequence that is 3 nucleotides in length is herein referred to as a 3-mer.

In some aspects, an N-mer sequence can be about 4 nucleotides in length. In some aspects, an N-mer sequence is 4 nucleotides in length. An N-mer sequence that is 4 nucleotides in length is herein referred to as a 4-mer.

In some aspects, an N-mer sequence can be about 5 nucleotides in length. In some aspects, an N-mer sequence is 5 nucleotides in length. An N-mer sequence that is 5 nucleotides in length is herein referred to as a 5-mer.

In some aspects, an N-mer sequence can be about 6 nucleotides in length. In some aspects, an N-mer sequence is 6 nucleotides in length. An N-mer sequence that is 6 nucleotides in length is herein referred to as a 6-mer.

In some aspects, an N-mer sequence can be any number of nucleotides in length. In some aspects, an N-mer sequence can be about at least 25 nucleotides, or at least about 50 nucleotides, or at least about 75 nucleotides, or at least about 100 nucleotides, or at least about 125 nucleotides, or at least about 150 nucleotides, or at least about 175 nucleotides, or at least about 200 nucleotides, or at least about 225 nucleotides, or at least about 250 nucleotides, or at least about 275 nucleotides, or at least about 300 nucleotides in length.

In some aspects, an N-mer sequence can be any number of nucleotides in length. In some aspects, an N-mer sequence can be about 25 nucleotides, or about 50 nucleotides, or about 75 nucleotides, or about 100 nucleotides, or about 125 nucleotides, or about 150 nucleotides, or about 175 nucleotides, or about 200 nucleotides, or about 225 nucleotides, or about 250 nucleotides, or about 275 nucleotides, or about 300 nucleotides in length.

In some aspects, an Addamer can comprise an MCS sequence, a first IISRE sequence, an N-mer sequence, and an at least second IISRE sequence. In some aspects, an Addamer can comprise an MCS sequence, followed by a first IISRE sequence, followed by an N-mer sequence, followed by an at least second IISRE sequence. An exemplary schematic of the preceding Addamer is shown in FIG. 8 as Addamer Designs #1-#4. In the non-limiting examples of Addamer Designs #1-#3 shown in FIG. 8, the first IISRE sequence is an IISRE sequence that when cleaved creates a 4 nucleotide long 5′ overhang, the N-mer sequence is a 3-mer sequence, and the at least second IISRE sequence is an IISRE sequence that when cleaved creates a blunt end. In the non-limiting example of Addamer Design #4 shown in FIG. 8, the first IISRE sequence is an IISRE sequence that when cleaved creates a blunt end, the N-mer sequence is a 3-mer sequence, and the at least second IISRE sequence is an IISRE sequence that when cleaved creates a 3 nucleotide long 5′ overhang.

In some aspects, an Addamer can comprise an MCS sequence, a first IISRE sequence, a second IISRE sequence, an N-mer sequence, a third IISRE sequence and at least fourth IISRE sequence. In some aspects, can comprise an MCS sequence, followed by a first IISRE sequence, followed by a second IISRE sequence, followed by an N-mer sequence, followed by a third IISRE sequence, followed by an at least fourth IISRE sequence. An exemplary schematic of the preceding Addamer is shown in FIG. 8 as Addamer Design #5. In the non-limiting example of Addamer Design #5 shown in FIG. 8, the first IISRE sequence is an IISRE sequence that when cleaved creates a 4 nucleotide long 5′ overhang, the second IISRE sequence is an IISRE sequence that when cleaved creates a 4 nucleotide long 5′ overhang, the N-mer sequence is a 3-mer sequence, the third IISRE sequence is an IISRE sequence that when cleaved creates a 4 nucleotide long 5′ overhang, and the at least fourth IISRE sequence is an IISRE sequence that when cleaved creates a blunt end.

In some aspects, an Addamer can comprise a first MCS sequence, a first IISRE sequence, an N-mer sequence, an at least second IISRE sequence and an at least second MCS sequence. In some aspects, an Addamer can comprise a first MCS sequence, followed by a first IISRE sequence, followed by an N-mer sequence, followed by an at least second IISRE sequence, followed by an at least second MCS sequence. An exemplary schematic of the preceding Addamer is shown in FIG. 8 as Addamer Design #6. In the non-limiting example of Addamer Design #6 shown in FIG. 8, the first IISRE sequence is an IISRE sequence that when cleaved creates a blunt end, the N-mer sequence is a 3-mer sequence, and the at least second IISRE sequence is an IISRE sequence that when cleaved creates a 4 nucleotide long 5-overhang.

In some aspects, an Addamer can comprise a first MCS sequence, a first IISRE sequence, a second IISRE sequence, an N-mer sequence, a third IISRE sequence, an at least fourth IISRE sequence and an at least second MCS sequence. In some aspects, an Addamer can comprise a first MCS sequence, followed by a first IISRE sequence, followed by a second IISRE sequence, followed by an N-mer sequence, followed by a third IISRE sequence, followed by an at least fourth IISRE sequence, followed by an at least second MCS sequence. An exemplary schematic of the preceding Addamer is shown in FIG. 8 as Addamer Design #7. In the non-limiting example of Addamer Design #7 shown in FIG. 8, the first IISRE sequence is an IISRE sequence that when cleaved creates a blunt end, the second IISRE sequence is an IISRE sequence that when cleaved creates a 4 nucleotide long 5′ overhang, the N-mer sequence is a 3-mer sequence, the third IISRE sequence is an IISRE sequence that when cleaved creates a 4 nucleotide long 5′ overhang, and that at least fourth IISRE sequence is an IISRE sequence that when cleaved creates a blunt end.

In some aspects, an Addamer can comprise a hairpin that comprises an aptamer sequence, a first IISRE sequence, an N-mer sequence, an at least second IISRE sequence and an MCS sequence. In some aspects, an Addamer can comprise a hairpin that comprises an aptamer sequence, followed by a first IISRE sequence, followed by an N-mer sequence, followed by an at least second IISRE sequence, followed by an MCS sequence. An exemplary schematic of the preceding Addamer is shown in FIG. 8 as Addamer Design #7. In the non-limiting example of Addamer Design #7 shown in FIG. 8, the aptamer sequence is a thrombin aptamer sequence, the first IISRE sequence is an IISRE sequence that when cleaved creates a 4 nucleotide long 5′ overhang, the N-mer sequence is a 3-mer sequence, and the at least second IISRE sequence is an IISRE that when cleaved creates a blunt end.

When two IISRE sequences are included adjacent to each other in an Addamer, these IISRE sequences can be called “Nested IISRE sequences” or “Nested IISRE sites”. In some aspects, nested IISRE sequences can comprise two IISRE sequences that are directly adjacent to one another. In some aspects, nested IISRE sequences can comprise two IISRE sequences that are adjacent to one another but separated by about 1 to about 10 nucleotides.

Without wishing to be bound by theory, it is possible to nest IISRE sites because some have cut sites sufficiently spaced from their recognition site as to fit other IISRE sites between the first site and the payload. Accordingly, in some aspects an Addamer can comprise a unique blunt cutter sites and a 4-base overhang sites on either side of the payload (N-mer sequence). Without wishing to be bound by theory, this significantly reduces the number of Addamer reagents needed to carry out routine nucleic acid generation.

Without wishing to be bound by theory, the inclusion of Nested IISRE sequence in an Addamer provides several options to cut at the same position with two distinct sites in the methods of the present disclosure. The option to cut at the same position with two distinct sites can reduce the number of distinct Addamers needed in a library (see below) for general nucleic acid synthesis.

In some aspects, Addamers can comprise any element known in the art to facilitate cloning, including but not limited to cognate sequences for amplification primers. Without wishing to be bound by theory, the inclusion of cognate sequences for amplification primers in an Addamer can allow for the recovery of specific Addamer designs for clonal propagation.

In some aspects, Addamers can comprise any element known in the art to facilitate large-scale production of the Addamer by fermentation in plasmids or bacteriophage. Such elements include, but are not limited to, sequences corresponding to DNAzyme scars and/or sequences that facilitate smooth folding of an Addamer after excision using certain DNAzymes (see e.g. Praetorius et al., Nature, 2017, 552, 84-87, incorporated herein by reference in its entirety).

### Nucleic Acid Synthesis Methods Using Addamers of the Present Disclosure

The Addamers described herein can be used in the methods described herein to synthesize a nucleic acid molecule comprising any target nucleic acid sequence. A target nucleic acid sequence is also referred to herein as a “target nucleic acid” or a “gene”.

In some aspects, a target nucleic acid sequence can be at least about 100, or at least about 200, or at least about 300, or at least about 500, or at least about 600, or at least about 700, or at least about 800, or at least about 900, or at least about 1000, or at least about 1500, or at least about 2000, or at least about 2500, or at least about 3000, or at least about 3500, or at least about 4000, or at least about 4500, or at least about 5000 nucleotides in length. In some aspects, the target double-stranded nucleic acid can comprise at least one homopolymeric sequence.

In some aspects, the target nucleic acid sequence can comprise at least one homopolymeric sequence. As used herein, the term homopolymeric sequence is used to refer to any type of repeating nucleic acid sequence, including, but not limited to, repeats of single nucleotides or repeats of small motifs. In some aspects, a homopolymeric sequence can be at least about 10 nucleotides, or at least about 20 nucleotides, or at least about 30 nucleotides, or at least about 40 nucleotides, or at least about 50 nucleotides, or at least about 60 nucleotides, or at least about 70 nucleotides, or at least about 80 nucleotides, or at least about 90 nucleotides, or at least about 100 nucleotides in length.

In some aspects, the target nucleic acid sequence can have a GC content of at least about 10%, or at least about 20%, or at least about 50%, or at least about.

As part of the synthesis methods of a present disclosure, one or more Addamers can be immobilized to a solid support. The solid support can be any solid support known in the art, including, but not limited to at least one bead. In some aspects, the at least one bead can comprise polyacrylamide, polystyrene, agarose or any combination thereof. In some aspects, the at least one bead can be magnetic. In some aspects, the solid support comprises a well or chamber. In some aspects, a solid support can comprise a plurality of wells or chambers. In some aspects, the plurality of wells comprises a multi-well plate. In some aspects, a solid support can comprise glass. In some aspects, a solid support can comprise a glass slide. In some aspects, a solid support can comprise quartz. In some aspects, a solid support can comprise a quartz slide. In some aspects, a solid support can comprise polystyrene. In some aspects, a solid support can comprise a polystyrene slide. In some aspects, a solid support can comprise a coating. wherein the coating prevents non-specific binding of unwanted proteins, unwanted nucleic acids or other unwanted biomolecules. In some aspects, a coating can comprise polyethylene glycol (PEG). In some aspects, a coating can comprise triethylene glycol (TEG).

In some aspects wherein an Addamer comprises a hairpin that comprises an aptamer sequence, the Addamer can be immobilized to a solid support via binding to the aptamer sequence. That is, the solid support can comprise at least one moiety that binds to the aptamer sequence on the Addamer. Accordingly, in a non-limiting example wherein an Addamer comprises a hairpin that comprises one of the aptamer sequences put forth in Table 3, the solid support can comprise the corresponding ligand listed in Table 3.

In some aspects wherein an Addamer comprises an MCS sequence, the Addamer can be immobilized to a solid support by a method comprising: a) contacting the Addamer with at least one corresponding restriction endonuclease to cleave the MCS sequence, thereby producing a 5′ overhang or a 3′ overhang; and b) hybridizing the 5′ overhang or 3′ overhang to a complementary single-stranded nucleic acid molecule on the solid support, thereby immobilizing the Addamer to the solid support. The preceding method can further comprise contacting the Addamer hybridized to the complementary single-stranded nucleic acid molecule on the solid support with a ligase, thereby ligating the Addamer and the complementary single-stranded nucleic acid molecule on the solid support.

In some aspects wherein an Addamer comprises an MCS sequence, the Addamer can be immobilized to a solid support by a method comprising: a) contacting the Addamer with at least one corresponding restriction endonuclease to cleave the MCS sequence, thereby producing a blunt end; and b) ligating the blunt end of the Addamer to a nucleic acid molecule located on the solid support, thereby immobilizing the Addamer to the solid support.

In some aspects, an Addamer that has been attached to a solid support can be referred to herein as an “attachment stud”.

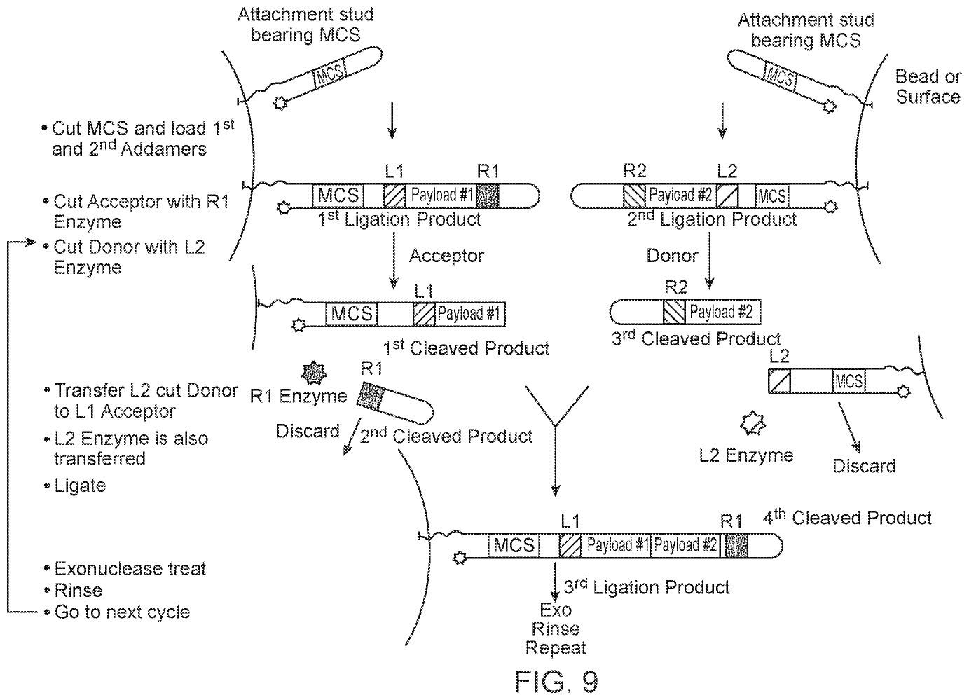

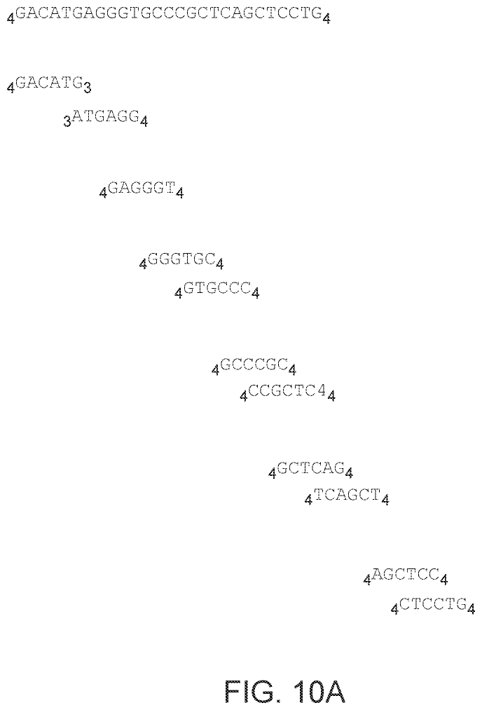

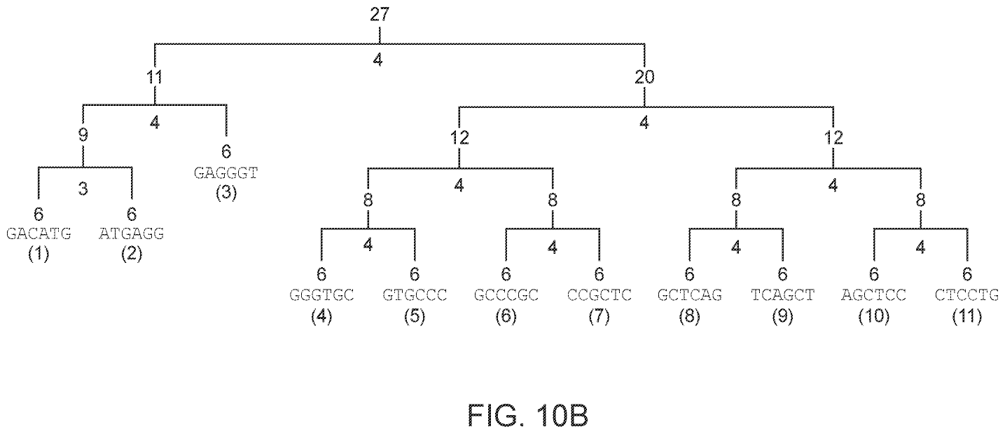

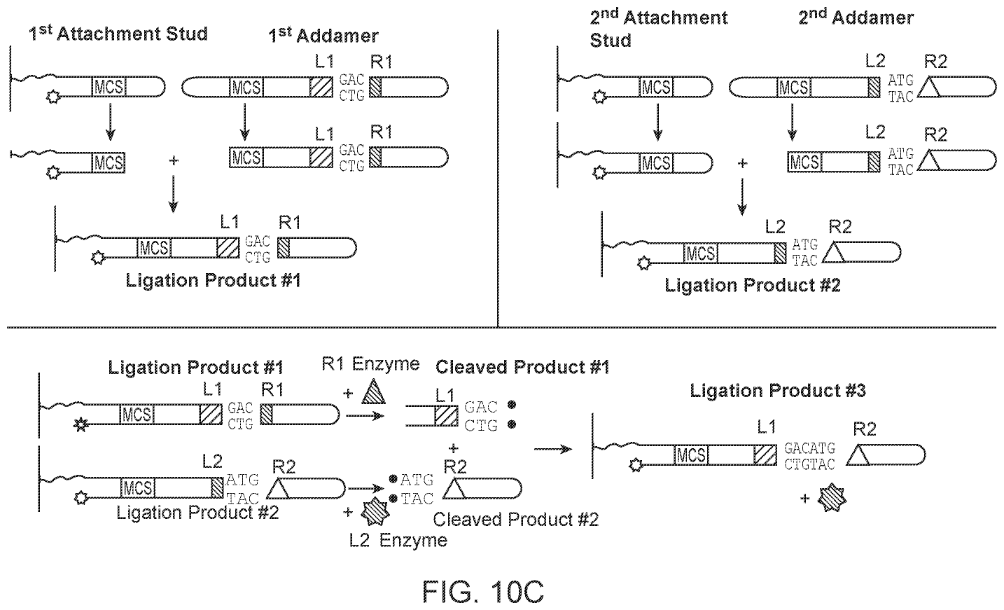

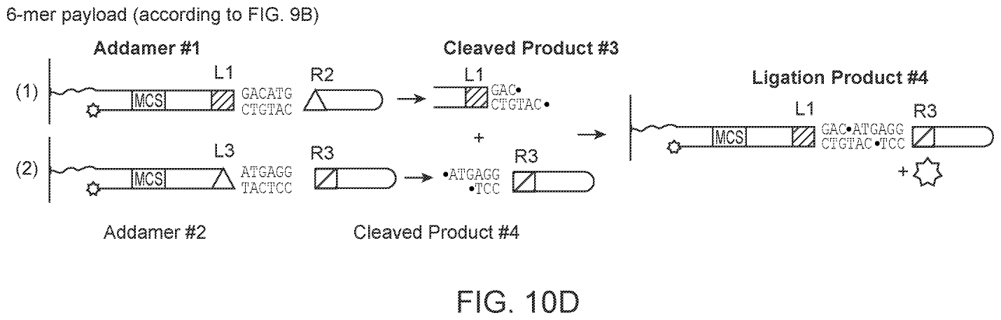

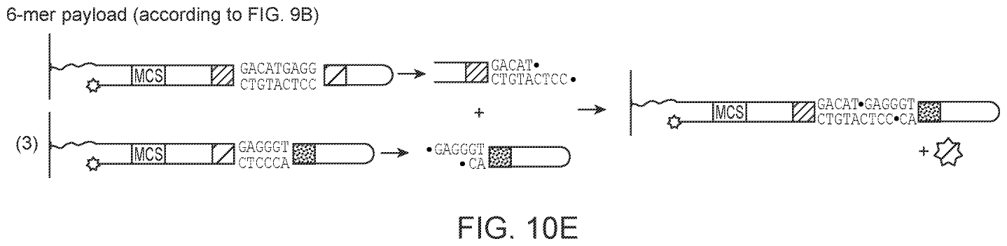

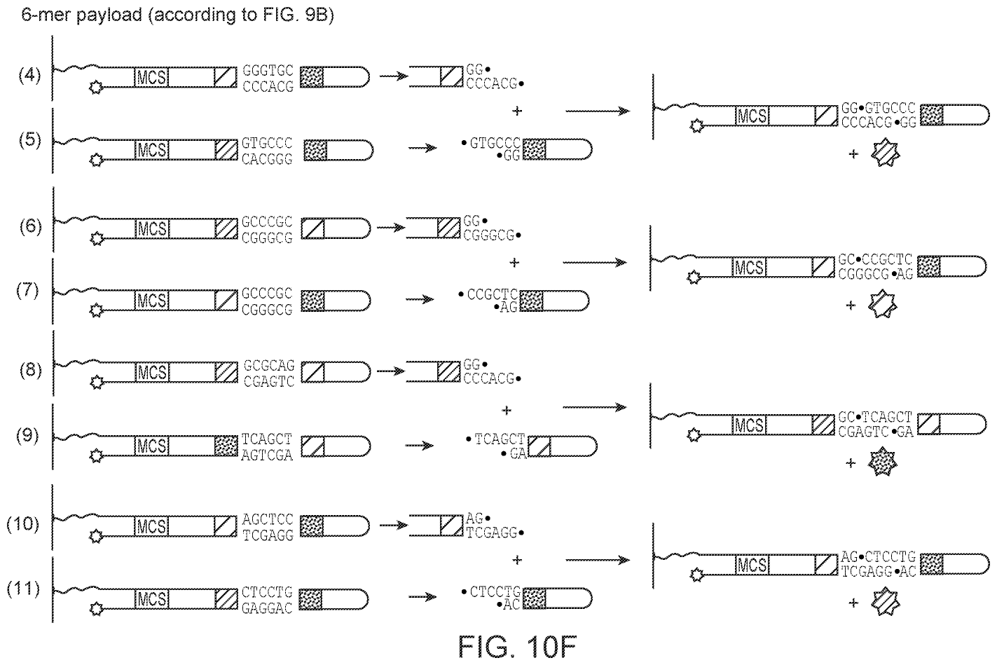

A schematic overview of the Addamer-based nucleic acid assembly/synthesis methods of the present disclosure is shown in FIG. 9.

In the first step of the method, an Addamer immobilized onto a solid support (denoted as a bead or surface in FIG. 9) is provided. This Addamer is herein referred to as an attachment stud and is connected at one end to the solid support using any of the methods described above and is capped at the other end with a hairpin. The attachment stud also comprises an MCS sequence. In the next step of the method, the attachment stud is contacted with a restriction endonuclease that cleaves the MCS sequence, thereby creating a 3′ overhang, a 5′ overhang or a blunt end. In the next step of the method, a first Addamer comprising an MCS sequence, a first IISRE sequence (denoted “L1” in FIG. 9), a first N-mer sequence (referred to as “Payload #1” in FIG. 9) and a second IISRE sequence (denoted “R1” in FIG. 9) is contacted with a restriction endonuclease that cleaves the MCS sequence, thereby creating a 3′ overhang, a 5′ overhang or a blunt end.

In the next step of the method, the cleaved first Addamer is ligated to the cleaved attachment stud by contacting the cleaved attachment stud, the cleaved Addamer and a ligase enzyme, thereby creating a first ligation product that is immobilized to the solid support and that comprises the MCS sequence, the first IISRE sequence, the Payload #1 sequence and the second IISRE sequence (see left side of FIG. 9). The first ligation product is then treated with exonuclease to remove any non-ligated attachment studs and/or first Addamers.

The steps described above are then repeated with another Addamer immobilized onto a solid support and a second Addamer comprising an MCS sequence, a third IISRE sequence (denoted “R2” in FIG. 9), a second N-mer sequence (referred to as “Payload #2” in FIG. 9) and a fourth IISRE sequence (denoted “L2” in FIG. 9) to produce a second ligation product that is immobilized to a solid support and that comprises an MCS sequence, the third IISRE sequence, the Payload #2 sequence and the fourth IISRE sequence (see right side of FIG. 9).

In the next step of the method, the 1^{st} ligation product is contacted with a IISRE (denoted “R1 enzyme” in FIG. 9) that cleaves the second IISRE sequence (R1), thereby creating a 3′ overhang, a 5′ overhang or a blunt end, thereby creating: a) a 1^{st} cleaved product that is immobilized to the solid support and that comprises the MCS sequence, the first IISRE sequence (R1), the Payload #1 sequence and a 3′ overhang, a 5′ overhang or a blunt end; and b) a 2^{nd} cleaved product comprising the second IISRE sequence (R1). The 2^{nd} cleaved product is then discarded by washing.

In the next step of the method, the 2^{nd} ligation product is contacted with a IISRE (denoted “L2 enzyme” in FIG. 9) that claves the fourth IISRE sequence (L2), thereby creating a 3′ overhang, a 5′ overhang or a blunt end, thereby creating: a) a 3^{rd} cleaved product that is released into solution and that comprises a hairpin at one end, the 3^{rd} IISRE sequence (R2), the Payload #2 sequence and a 3′ overhang, a 5′ overhang or a blunt end; and b) a 4th cleaved product that is immobilized to the solid support and that comprises the MCS sequence and the fourth IISRE sequence (L2).

In the next step, the 1^{st} cleaved product and the 3^{rd} cleaved product are ligated together by contacting the 1^{st} cleaved product, the 3^{rd} cleaved product and a ligase enzyme (e.g. the solution comprising the 3^{rd} cleaved product is transferred to the solution comprising the 1^{st} cleaved product immobilized to the solid surface and a ligase enzyme is added to the solution), thereby creating a 3^{rd} ligation product that is immobilized to a solid surface and that comprises an MCS sequence, the first IISRE sequence (L1), the Payload #1 sequence, the Payload #2 sequence and the third IISRE sequence (R2). This ligation reaction is then treated with exonuclease to remove any non-ligated 1^{st} Cleaved Products and/or 3^{rd} Cleaved Products.

The steps described above can be repeated until a target nucleic acid sequence is synthesized.

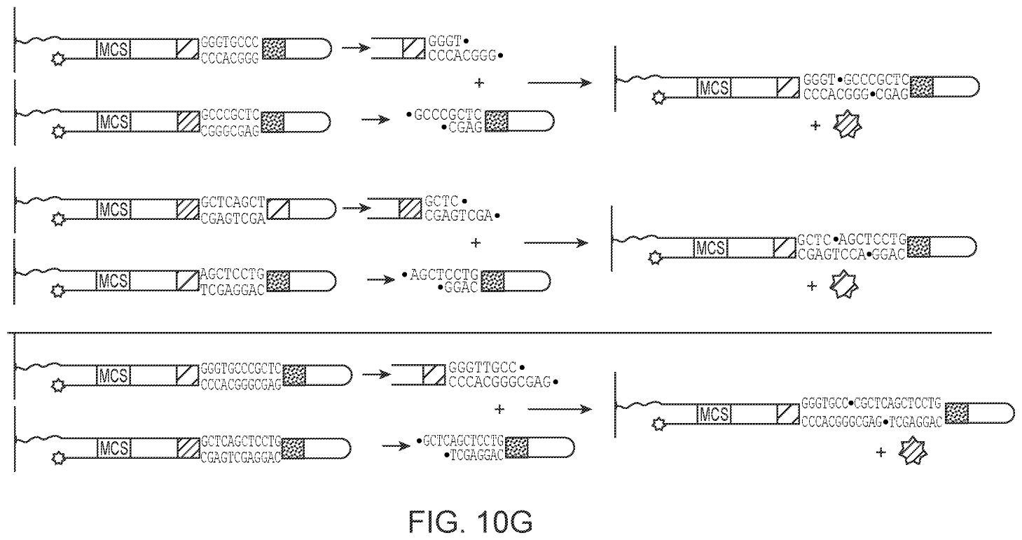

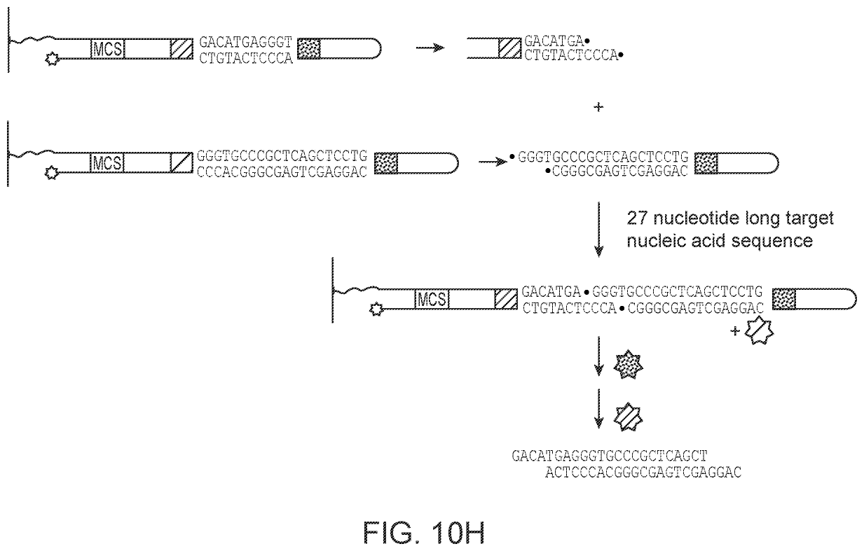

A schematic overview of the synthesis of an exemplary 27-nucleotide long target nucleic acid sequence is shown in FIG. 10A-10H. The sequence to be synthesized is shown at the top of FIG. 10A. The sequence is subdivided into eleven, 6-mer fragments that overlap by either 3 nucleotides or 4 nucleotides that are to be incorporated into Addamers that are to be ligated together to synthesize the target nucleic acid sequence. FIG. 10B shows an assembly tree for the exemplary target nucleic acid sequence that maps the order in which the Addamers comprising the 6-mer fragments are to be ligated to efficiently synthesize the target nucleic acid sequence. While there are several different ways to traverse the assembly and placement of odd versus even overhangs, the assembly order is to be dictated by the compatibility of IISRE enzyme sites with the sequences to be generated. In FIG. 10B the numbered 6-mers (1)-(11) correspond to the numbered 6-mers in FIGS. 10C-10H. The numbers at each node of the tree correspond to the payload length at each step of the assembly. The ‘4’ and ‘3’ indicate the length of the overhang used. The length of a resulting payload sequence is, length=a+b−n, where ‘a’ and ‘b’ are the lengths of the input payloads and ‘n’ is the length of the overhang.

The first step of the synthesis of the target nucleic acid sequence is shown in FIG. 9C, which depicts the loading of an Addamer comprising an 3-mer sequence of GAC and an Addamer comprising an 3-mer sequence of ATC to form an Addamer comprising a GACATG 6-mer, which is 6-mer #1 in FIG. 10B. To produce the GACATG hexamer, a first attachment stud comprising an MCS sequence and a first Addamer comprising an MCS sequence, a first IISRE sequence (denoted “L1” in FIG. 10C), a 3-mer sequence comprising the sequence GAC, and a second IISRE sequence (denoted “R1” in FIG. 10C) are contacted with one or more restriction endonucleases to cleave the MCS sequences, thereby creating complementary overhangs. These complementary overhangs are then hybridized and the Addamer and the attachment stud are ligated together by contacting the hybridized complex with a ligase enzyme to yield Ligation Product #1 that is immobilized to the solid surface and that comprises the MCS sequence, the first IISRE sequence (L1), the 3-mer sequence GAC, and the second IISRE sequence (R1). The same process can be repeated with a second attachment stud comprising an MCS sequence and a second Addamer comprising an MCS sequence, a third IISRE sequence (denoted “L2” in FIG. 10C), the 3-mer sequencing comprising the sequence ATG, and a fourth IISRE sequence (denoted “R2” in FIG. 10C) to yield Ligation Product #2 that is immobilized to the solid surface and that comprises the MCS sequence, the third IISRE sequence (L2), the 3-mer sequence ATG and the fourth IISRE sequence (R2). Next, Ligation Product #1 is contacted with a IISRE (denoted “R1 enzyme” in FIG. 10C) that cleaves the second IISRE sequence (R1), thereby producing Cleaved Product #1 that is immobilized to the solid surfaces and that comprises the MCS sequence, the first IISRE sequence (L1) and the 3-mer sequence GAC followed by a blunt end. Similarly, Ligation Product #2 is contacted with a IISRE (denoted “L2 enzyme” in FIG. 10C) that cleaves the third IISRE sequence (L2), thereby producing Cleaved Product #2 that is released into solution and that comprises the a blunt end, the 3-mer sequence ATG and the fourth IISRE sequence (R2). Cleaved Product #1 and Cleaved Product #2 are then ligated together using a ligase enzyme to yield Ligation Product #3 that is immobilized to the solid support and that comprises the MCS sequence, the first IISRE sequence (L1), the 6-mer sequence GACATG and the fourth IISRE sequence (R2). These products can optionally be treated with exonuclease to remove any non-ligated Cleaved Product #1 and/or Cleaved Product #2. The steps described in this paragraph can be repeated with additional Addamers comprising different 3-mer sequences to generate the Addamers comprising 6-mer sequences #2-#11 shown in FIG. 10B.

The method continues in FIG. 10D, which shows the ligation of Addamers comprising 6-mer Sequence #1 and 6-mer Sequence #2 (see FIG. 10B). Addamer #1 is immobilized to a solid surface and comprises an MCS sequence, the first IISRE site (L1) from FIG. 10C, the 6-mer sequence GACATG (6-mer sequence #1 from FIG. 10B), and the fourth IISRE sequence (R2) from FIG. 10C. Addamer #2 is immobilized to a solid surface and comprises an MCS sequence, a fifth IISRE site (denoted “L3” in FIG. 10D), the 6-mer sequence ATGAGG (6-mer sequence #2 from FIG. 10B) and a sixth IISRE site (denoted “R3” in FIG. 10D). Addamer #1 is contacted with a IISRE that cleaves the fourth IISRE site (R2) to produce a single-stranded overhang in the N-mer sequence, thereby producing Cleaved Product #3 that is immobilized to the solid surface and that comprises the MCS sequence, the first IISRE sequence (L1), and the N-mer sequence with a single-stranded overhang. Addamer #2 is contacted with a IISRE that claves the fifth IISRE site (L3) to produce a single-stranded overhang in the N-mer sequence, thereby producing Cleaved Product #4 that is released into solution and the comprises the N-mer sequence with a single-stranded overhang and the sixth IISRE sequence (R3). Cleaved Product #3 and Cleaved Product #4 are then ligated together using a ligase enzyme to yield ligation Product #4 that is immobilized to the solid surface and that comprises the MCS sequence, the first IISRE sequence (L1), the N-mer sequence GACATGAGG (the first nine nucleotides in the target nucleic acid sequence to be synthesized) and the sixth IISRE sequence (R3). Ligation Product #4 can optionally be treated with exonuclease to remove any non-ligated Cleaved Product #1 and/or Cleaved Product #2.

The method continues in FIG. 10E, where Ligation Product #4 and an Addamer that comprises 6-mer Sequence #3 (see FIG. 10B) are treated with corresponding IISREs to produce cleaved products that are then ligated together to produce an Addamer that is immobilized to the solid surface and that comprises the N-mer sequence GACATGAGGGT (SEQ ID NO: 116), the first 11 nucleotides in the target nucleic acid sequence to be synthesized.

The sequential IISRE digestions and ligations are repeated in FIGS. 10F-10H according the assembly map shown in FIG. 10B until an Addamer comprising an N-mer sequence that corresponds to the 27 nucleotide long target nucleic acid sequence is synthesized. In a final step, the 27 nucleotide long target nucleic acid sequence can be excised from the final synthesized Addamer by treating the Addamer with IISREs that cleave the IISRE sequences that flank the 27 nucleotide long target nucleic acid sequence.

The preceding methods can be described as follows: a) providing a first Addamer of the present disclosure that is immobilized to a solid support, wherein the first Addamer comprises a first IISRE sequence, followed by a first N-mer sequence, followed by a second IISRE sequence, followed by a hairpin structure; b) providing a second Addamer of the present disclosure that is immobilized to a solid support, wherein the second Addamer comprising a third IISRE sequence, followed by a second N-mer sequence, followed by a fourth IISRE sequence, followed by a hairpin structure; c) contacting the first Addamer with a IISRE that cleaves the second IISRE sequence located in the first Addamer, thereby producing a first Cleaved Product that is immobilized to the solid support and that comprises the first IISRE sequence, the first N-mer sequence and at least one of a 3′ overhang, a 5′ overhang and a blunt end; d) contacting the second Addamer with a IISRE that cleaves the third IISRE sequence located in the second Addamer, thereby producing a second Cleaved Product that is released into solution and that comprises the second N-mer sequence, the fourth IISRE sequence, and at least one of a 3′ overhang, a 5′ overhang and a blunt end, and wherein the second Cleaved Product is capped at one end by a hairpin structure; e) ligating the first Cleaved Product and the Second Cleaved Product using a ligase enzyme to produce a first Ligation Product; f) treating the products of step (e) with an exonuclease, thereby removing non-ligated first Cleaved Product and/or second Cleaved Product; and g) repeating steps (a)-(f) until the nucleic acid molecule comprising the target nucleic acid sequence has been synthesized.

The preceding methods can be described as follows: a method comprising: method comprising: a) providing a first Addamer of the present disclosure that is immobilized to a solid support, wherein the first Addamer comprises a first IISRE sequence, followed by a first N-mer sequence, followed by a second IISRE sequence, followed by a hairpin structure; b) providing a second Addamer of the present disclosure that is immobilized to a solid support, wherein the second Addamer comprising a third IISRE sequence, followed by a second N-mer sequence, followed by a fourth IISRE sequence, followed by a hairpin structure; c) contacting the first Addamer with a IISRE that cleaves the second IISRE sequence located in the first Addamer, thereby producing a first Cleaved Product that is immobilized to the solid support and that comprises the first IISRE sequence, the first N-mer sequence and at least one of a 3′ overhang, a 5′ overhang and a blunt end; d) contacting the second Addamer with a IISRE that cleaves the third IISRE sequence located in the second Addamer, thereby producing a second Cleaved Product that is released into solution and that comprises the second N-mer sequence, the fourth IISRE sequence, and at least one of a 3′ overhang, a 5′ overhang and a blunt end, and wherein the second Cleaved Product is capped at one end by a hairpin structure; e) ligating the first Cleaved Product and the Second Cleaved Product using a ligase enzyme to produce a first Ligation Product; f) treating the products of step (e) with an exonuclease, thereby removing non-ligated first Cleaved Product and/or second Cleaved Product; and g) repeating steps (c)-(f) until the nucleic acid molecule comprising the target nucleic acid sequence has been synthesized.

The preceding methods can be described as follows: a) providing a first Addamer of the present disclosure that is immobilized to a solid support, wherein the first Addamer comprises a first IISRE sequence, followed by a first N-mer sequence, followed by a second IISRE sequence, followed by a hairpin structure; b) providing a second Addamer of the present disclosure that is immobilized to a solid support, wherein the second Addamer comprising a third IISRE sequence, followed by a second N-mer sequence, followed by a fourth IISRE sequence, followed by a hairpin structure; c) contacting the first Addamer with a IISRE that cleaves the second IISRE sequence located in the first Addamer, thereby producing a first Cleaved Product that is immobilized to the solid support and that comprises the first IISRE sequence, the first N-mer sequence and at least one of a 3′ overhang, a 5′ overhang and a blunt end; d) contacting the second Addamer with a IISRE that cleaves the third IISRE sequence located in the second Addamer, thereby producing a second Cleaved Product that is released into solution and that comprises the second N-mer sequence, the fourth IISRE sequence, and at least one of a 3′ overhang, a 5′ overhang and a blunt end, and wherein the second Cleaved Product is capped at one end by a hairpin structure; e) ligating the first Cleaved Product and the Second Cleaved Product using a ligase enzyme to produce a first Ligation Product; f) treating the products of step (e) with an exonuclease, thereby removing non-ligated first Cleaved Product and/or second Cleaved Product; and g) repeating steps (a)-(f) with one or more additional Addamers until the nucleic acid molecule comprising the target nucleic acid sequence has been synthesized.

The preceding methods can be described as follows: a method comprising: method comprising: a) providing a first Addamer of the present disclosure that is immobilized to a solid support, wherein the first Addamer comprises a first IISRE sequence, followed by a first N-mer sequence, followed by a second IISRE sequence, followed by a hairpin structure; b) providing a second Addamer of the present disclosure that is immobilized to a solid support, wherein the second Addamer comprising a third IISRE sequence, followed by a second N-mer sequence, followed by a fourth IISRE sequence, followed by a hairpin structure; c) contacting the first Addamer with a IISRE that cleaves the second IISRE sequence located in the first Addamer, thereby producing a first Cleaved Product that is immobilized to the solid support and that comprises the first IISRE sequence, the first N-mer sequence and at least one of a 3′ overhang, a 5′ overhang and a blunt end; d) contacting the second Addamer with a IISRE that cleaves the third IISRE sequence located in the second Addamer, thereby producing a second Cleaved Product that is released into solution and that comprises the second N-mer sequence, the fourth IISRE sequence, and at least one of a 3′ overhang, a 5′ overhang and a blunt end, and wherein the second Cleaved Product is capped at one end by a hairpin structure; e) ligating the first Cleaved Product and the Second Cleaved Product using a ligase enzyme to produce a first Ligation Product; f) treating the products of step (e) with an exonuclease, thereby removing non-ligated first Cleaved Product and/or second Cleaved Product; and g) repeating steps (c)-(f) with one or more additional addamers until the nucleic acid molecule comprising the target nucleic acid sequence has been synthesized.

The preceding methods can be described as follows: a) providing a first Addamer of the present disclosure that is immobilized to a solid support, wherein the first Addamer comprises a first IISRE sequence, followed by a first N-mer sequence, followed by a second IISRE sequence, followed by a hairpin structure; b) providing a second Addamer of the present disclosure that is immobilized to a solid support, wherein the second Addamer comprising a third IISRE sequence, followed by a second N-mer sequence, followed by a fourth IISRE sequence, followed by a hairpin structure; c) contacting the first Addamer with a IISRE that cleaves the second IISRE sequence located in the first Addamer, thereby producing a first Cleaved Product that is immobilized to the solid support and that comprises the first IISRE sequence, the first N-mer sequence and at least one of a 3′ overhang, a 5′ overhang and a blunt end; d) contacting the second Addamer with a IISRE that cleaves the third IISRE sequence located in the second Addamer, thereby producing a second Cleaved Product that is released into solution and that comprises the second N-mer sequence, the fourth IISRE sequence, and at least one of a 3′ overhang, a 5′ overhang and a blunt end, and wherein the second Cleaved Product is capped at one end by a hairpin structure; e) ligating the first Cleaved Product and the Second Cleaved Product using a ligase enzyme to produce a first Ligation Product; f) treating the products of step (e) with an exonuclease, thereby removing non-ligated first Cleaved Product and/or second Cleaved Product; and g) repeating steps (a)-(f) using the products of step (f) and one or more additional Addamers until the nucleic acid molecule comprising the target nucleic acid sequence has been synthesized.

The preceding methods can be described as follows: a method comprising: method comprising: a) providing a first Addamer of the present disclosure that is immobilized to a solid support, wherein the first Addamer comprises a first IISRE sequence, followed by a first N-mer sequence, followed by a second IISRE sequence, followed by a hairpin structure; b) providing a second Addamer of the present disclosure that is immobilized to a solid support, wherein the second Addamer comprising a third IISRE sequence, followed by a second N-mer sequence, followed by a fourth IISRE sequence, followed by a hairpin structure; c) contacting the first Addamer with a IISRE that cleaves the second IISRE sequence located in the first Addamer, thereby producing a first Cleaved Product that is immobilized to the solid support and that comprises the first IISRE sequence, the first N-mer sequence and at least one of a 3′ overhang, a 5′ overhang and a blunt end; d) contacting the second Addamer with a IISRE that cleaves the third IISRE sequence located in the second Addamer, thereby producing a second Cleaved Product that is released into solution and that comprises the second N-mer sequence, the fourth IISRE sequence, and at least one of a 3′ overhang, a 5′ overhang and a blunt end, and wherein the second Cleaved Product is capped at one end by a hairpin structure; e) ligating the first Cleaved Product and the Second Cleaved Product using a ligase enzyme to produce a first Ligation Product; f) treating the products of step (e) with an exonuclease, thereby removing non-ligated first Cleaved Product and/or second Cleaved Product; and g) repeating steps (c)-(f) using the products of step (f) and one or more additional Addamers until the nucleic acid molecule comprising the target nucleic acid sequence has been synthesized.

The preceding methods can be described as follows: a) providing a first Addamer of the present disclosure that is immobilized to a solid support, wherein the first Addamer comprises a first IISRE sequence, followed by a first N-mer sequence, followed by a second IISRE sequence, followed by a hairpin structure; b) providing a second Addamer of the present disclosure that is immobilized to a solid support, wherein the second Addamer comprising a third IISRE sequence, followed by a second N-mer sequence, followed by a fourth IISRE sequence, followed by a hairpin structure; c) contacting the first Addamer with a IISRE that cleaves the second IISRE sequence located in the first Addamer, thereby producing a first Cleaved Product that is immobilized to the solid support and that comprises the first IISRE sequence, the first N-mer sequence and at least one of a 3′ overhang, a 5′ overhang and a blunt end; d) contacting the second Addamer with a IISRE that cleaves the third IISRE sequence located in the second Addamer, thereby producing a second Cleaved Product that is released into solution and that comprises the second N-mer sequence, the fourth IISRE sequence, and at least one of a 3′ overhang, a 5′ overhang and a blunt end, and wherein the second Cleaved Product is capped at one end by a hairpin structure; e) ligating the first Cleaved Product and the Second Cleaved Product using a ligase enzyme to produce a first Ligation Product; f) treating the products of step (e) with an exonuclease, thereby removing non-ligated first Cleaved Product and/or second Cleaved Product; and g) repeating any combination of steps (a)-(f) using the products of step (f) and/or one or more additional Addamers until the nucleic acid molecule comprising the target nucleic acid sequence has been synthesized.

The preceding methods can be described as follows: a method comprising: method comprising: a) providing a first Addamer of the present disclosure that is immobilized to a solid support, wherein the first Addamer comprises a first IISRE sequence, followed by a first N-mer sequence, followed by a second IISRE sequence, followed by a hairpin structure; b) providing a second Addamer of the present disclosure that is immobilized to a solid support, wherein the second Addamer comprising a third IISRE sequence, followed by a second N-mer sequence, followed by a fourth IISRE sequence, followed by a hairpin structure; c) contacting the first Addamer with a IISRE that cleaves the second IISRE sequence located in the first Addamer, thereby producing a first Cleaved Product that is immobilized to the solid support and that comprises the first IISRE sequence, the first N-mer sequence and at least one of a 3′ overhang, a 5′ overhang and a blunt end; d) contacting the second Addamer with a IISRE that cleaves the third IISRE sequence located in the second Addamer, thereby producing a second Cleaved Product that is released into solution and that comprises the second N-mer sequence, the fourth IISRE sequence, and at least one of a 3′ overhang, a 5′ overhang and a blunt end, and wherein the second Cleaved Product is capped at one end by a hairpin structure; e) ligating the first Cleaved Product and the Second Cleaved Product using a ligase enzyme to produce a first Ligation Product; f) treating the products of step (e) with an exonuclease, thereby removing non-ligated first Cleaved Product and/or second Cleaved Product; and g) repeating any combination of steps (c)-(f) using the products of step (f) and/or one or more additional Addamers until the nucleic acid molecule comprising the target nucleic acid sequence has been synthesized.

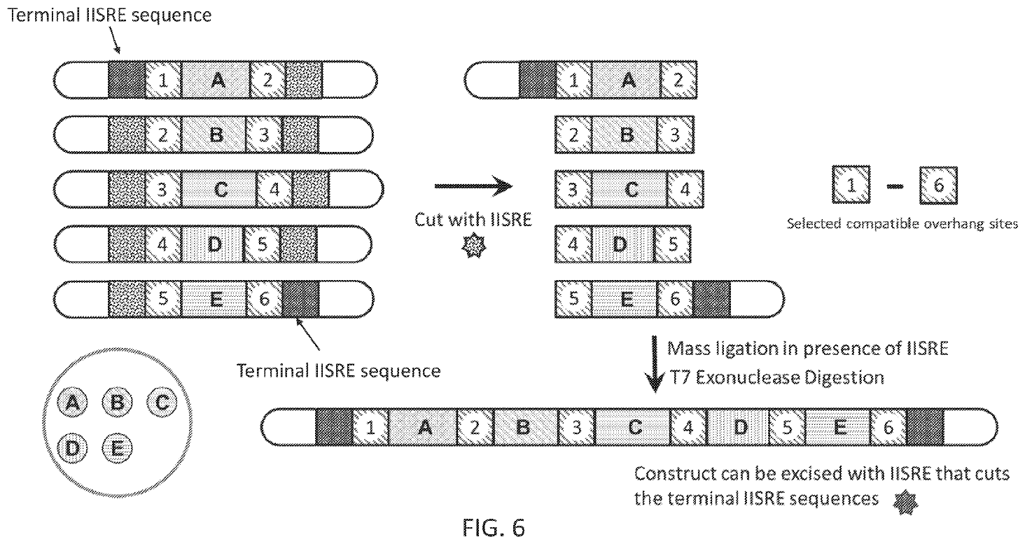

Another Addamer-based gene synthesis method, referred to as the “pooled synthesis method” is shown schematically in FIG. 6. As shown in FIG. 6, in a pooled synthesis method, a plurality of Addamers comprising a plurality of different Addamer species are provided in a common volume. Each of the Addamers species comprise a hairpin, followed by a first IISRE sequence, followed by a payload sequence, followed by a second IISRE sequence, followed by a hairpin. Two of the Addamer species comprise a “terminal IISRE sequence” that is cleaved at the end of the method to release the fully-assembled/synthesized target nucleic acid sequence. A schematic of these Addamer species is shown in FIG. 6. The IISRE sequences are shown as dotted boxes, with the terminal IISRE sequences specifically labeled. The payload sequences are denoted “A”, “B”, “C”, “D” and “E”. That is, in the non-limiting example shown in FIG. 6, the target gene has been divided into five fragments for purposes of the assembly process. After providing the plurality of Addamers in a common volume, the Addamers are contacted with one or more IISRE that cleaves each of the IISRE sequences except for the terminal IISRE sequences, resulting in the creation of single-stranded overhang sites, which are denoted in FIG. 6 by striped boxes labeled “1”, “2”, “3”, “4”, “5” and “6”. In the next step, complementary single-stranded overhang sites are hybridized together and the resulting hybridized complex is contacted with a ligase to ligate the digested Addamers together. As shown in FIG. 6, this ligation step can be performed in the presence of the IISREs. Following ligation, the ligation product can be treated with T7 exonuclease to remove any nonligated Addamers. The result of the ligation and T7 exonuclease digestion steps is shown in the bottom of FIG. 6—namely, the target nucleic acid sequence (“A-B-C-D-E”) is completely assembled and flanked by hairpins on either side. The target nucleic acid sequence can then be excised by this product with one or more IISREs that cleave the terminal IISRE sequences.

In the pooled synthesis method, the IISREs and IISRE sequences that are chosen are designed to create a set of complementary single-stranded overhangs that result in the assembly of the target nucleic acid sequence following hybridization of the single-stranded overhang sequences. In some aspects of the pooled synthesis method, the number of available IISRE sequences in a plurality of Addamers to be used to assemble a target nucleic acid is two, wherein the first IISRE sequence is used to achieve the initial assembly and second IISRE sequence is to release the assembled gene for subsequent cloning in plasmids or bacteriophage. Additional considerations in design are the overall length of the assembly as well as the lengths of each fragment. The sequence adjacent to the site need to be considered as there may be instances of the formation of secondary structures, such as G-quadruplexes, that interfere with ligation. The number of fragments used to assemble the gene is also an important consideration. A gene assembly of 400 bp, for example, would only require two Addamer payloads of 200 bp each. Likewise, a 1 kb gene would require at least five Addamer payloads of 200 bp.

Accordingly, the present disclosure provides a method of synthesizing a nucleic acid molecule comprising a target nucleic acid sequence, wherein the target nucleic acid sequence has been subdivided into two or more sequence fragments, the method comprising: a) providing a plurality of Addamers, wherein the plurality of Addamers comprises a plurality of different Addamer species, wherein each of the Addamer species comprise: a hairpin structure, followed by a first IISRE sequence, followed by a payload sequence, followed by a second IISRE sequence, followed by a hairpin structure, wherein the payload sequence of an Addamer species corresponds to one of the sequence fragments of the target nucleic acid sequence, wherein the plurality of Addamers comprises at least one Addamer for every sequence fragment; b) contacting the plurality of Addamers with at least one IISRE that cleaves at least one IIRSRE sequence in each of the Addamers, thereby producing at least one single-stranded overhang; c) ligating together the cleaved Addamers by contacting the cleaved Addamers with at least one ligase enzyme, thereby synthesizing the nucleic acid molecule comprising the target nucleic acid sequence. In some aspects, the preceding method can further comprise a MutS enzyme treatment step. In some aspects, the preceding method can further comprise treating the products of step (c) with an exonuclease, thereby purifying properly ligated products.

The Addamer design for the synthesis of a particular target nucleic acid entails the analysis of the sequence to be assembled/synthesized to ensure that the sites the IISREs are not present and second to choose the most appropriate junction site for ligation of the individual gene fragments. The junction sites derive from a collection of compatibility groups, which have been discovered in the process of developing the gSynth assembly process. Compatibility groups can be as few as five 4-base 5′ overhang sites (see PCT Application No. PCT/US2020/051838, published as WO2021055962A1) and as many as 35 4-base 5′ overhang sites (Potapov V et al., Comprehensive Profiling of Four Base Overhang Ligation Fidelity by T4 DNA Ligase and Application to DNA Assembly. ACS Synth. Biol. 2018, 7, 11, 2665-2674 and Pryor J. M. et al., Enabling one-pot Golden Gate assemblies of unprecedented complexity using data-optimized assembly design. PLOS ONE 15(9): e0238592. 2020), which have been used in Golden Gate assemblies.

In some aspects of the methods of the present disclosure, a ligase enzyme can be a human DNA ligase III (hLig3). As would be appreciated by the skilled artisan, hLig3 exhibits high blunt end ligation efficiency (>60%). In some aspects of the methods of the present disclosure, a ligase enzyme can be a T4 DNA ligase. As would be appreciated by the skilled artisan, T4 DNA ligase exhibits high ligation efficiency of nucleic acid fragments comprising 2, 3 or 4-nucleotide long 3′ or 5′ overhangs (>80%). A ligase enzyme can be any ligase enzyme known in the art.

In some aspects of the methods of the present disclosure, the exonuclease can be T7 exonuclease.

It has been demonstrated that modified Cas9 enzymes can be used to generate nicks instead of full double stranded breaks (Mali P et al., CAS9 transcriptional activators for target specificity screening and paired nickases for cooperative genome engineering. Nature Biotechnology volume 31, p833-838, 2013, and Ann Ran, F. et al., Double Nicking by RNA-Guided CRISPR Cas9 for Enhanced Genome Editing Specificity. Cell, volume 154, issue 6, p1380-1389, Sep. 12, 2013). Such Cas9 nickase activity can be directed to specific sites in target DNA by single guide RNAs (sgRNA). Additionally, this nickase activity can be directed to distinct plus and minus DNA strands in an opposing fashion, leading to nicks that expose overhanging single stranded DNA after digestion. A study of such opposing nicking site shows that a 10-15 base overhang produced best ligation results, interestingly a 10 base pair spacing is the distance of one turn of the double helix (Wang. R. Y. et al., DNA Fragments Assembly Based on Nicking Enzyme System. PLOS One, March 2013, Volume 8, Issue 3, e57943). Accordingly, in some aspects of Addamer-based synthesis methods, a mutant Cas9 combined with sgRNA targeting the ends of the payloads within Addamers can be used to generate overhangs that can be ligated to assemble the gene of interest rather than IIRE sequences and IIREs.

In some aspects of the methods of the present disclosure, the synthesized target nucleic acid sequence has a purity of at least about 80%, or at least about 85%, or at least about 90%, or at least about 95%, or at least about 99%.

In some aspects, the purity of a synthesized target nucleic acid sequence refers to the percentage of the total ligation products that were formed as part of a single ligation reaction, or multiple rounds of ligation reactions, that correspond to the correct/desired ligation product. Without wishing to be bound by theory, the methods of the present disclosure comprising the ligation of nuclei acid molecules produce can produce plurality of ligation products, some of which correspond to the correct/desired ligation product, and some that are undesired (side-reactions, incorrect ligations, etc.). The purity of a ligation product, or a target molecule that is being synthesized, can be expressed as a percentage, which corresponds to the percentage of the total ligation products formed which correspond to the correct/desired ligation product.

### gSynth Methods and Compositions of the Present Disclosure

The pooled oligonucleotide synthesis methods and compositions disclosed herein can be used in combination with gSynth methods, which are described in detail in PCT Application No. PCT/US2020/051838, published as WO2021055962A1. gSynth methods are also referred to as “double-stranded geometric synthesis (gSynth)” and compositions related thereto for the synthesis of long, arbitrary double-stranded nucleic acid sequences. In a double-stranded gSynth assembly reaction, the target sequence (i.e. the sequence that is to be synthesized) is computationally broken into a sets of adjacent, double-stranded nucleic acid fragments, These adjacent double-stranded nucleic acid fragments are then ligated together in one-pair at-a-time ligation reactions in a systematic assembly method. These fragments possess 3′ and/or 5′ overhanging single-stranded N-mer sites, with three properties: 1) The N-mer sites are not self-hybridizing or self-reactive in ligation reactions. 2) The N-mer site at one end of the fragment does not cross-hybridize or cross-react with the N-mer site at the other end. Finally, 3) there is one N-mer site on each fragment of an adjacent pair of fragments in that will hybridize and ligate with the adjacent fragment in a ligation reaction leading to a new, longer double-stranded fragment. PCT Application No. PCT/US2020/051838, published as WO2021055962A1, provides preferred N-mer sites that facilitate more efficient and accurate ligation reactions, thereby allowing the double-stranded gSynth methods of the present disclosure to be used to synthesize nucleic acid sequences of unprecedented lengths that are not achievable using existing nucleic acid assembly and synthesis techniques. The double-stranded fragments of the present disclosure can be generated using the methods described herein.

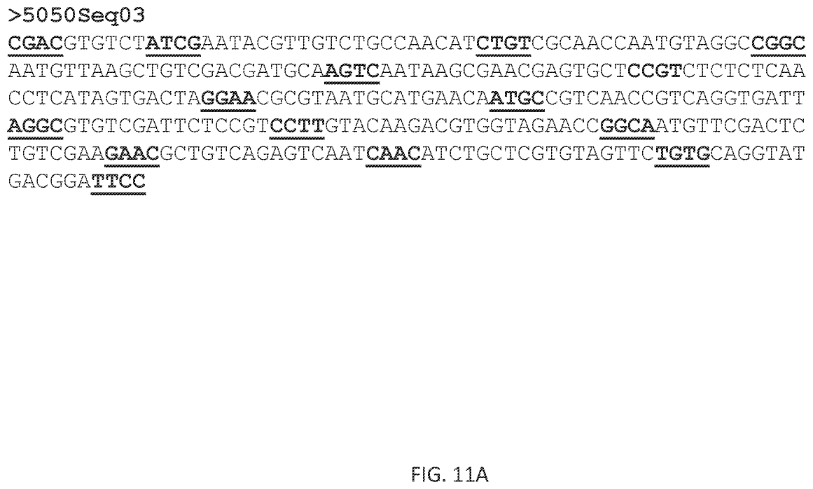

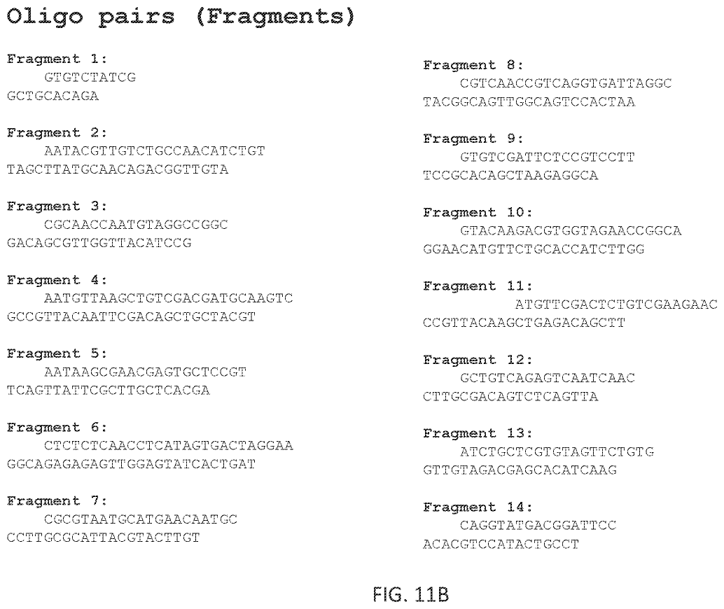

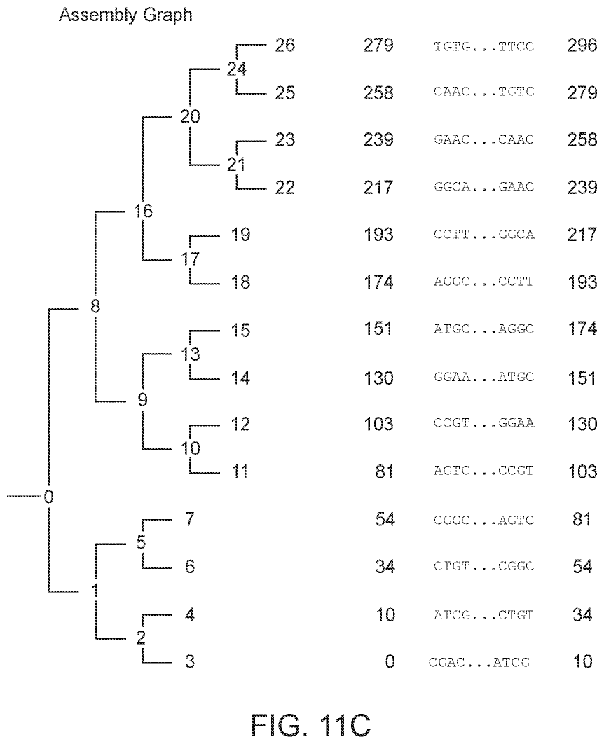

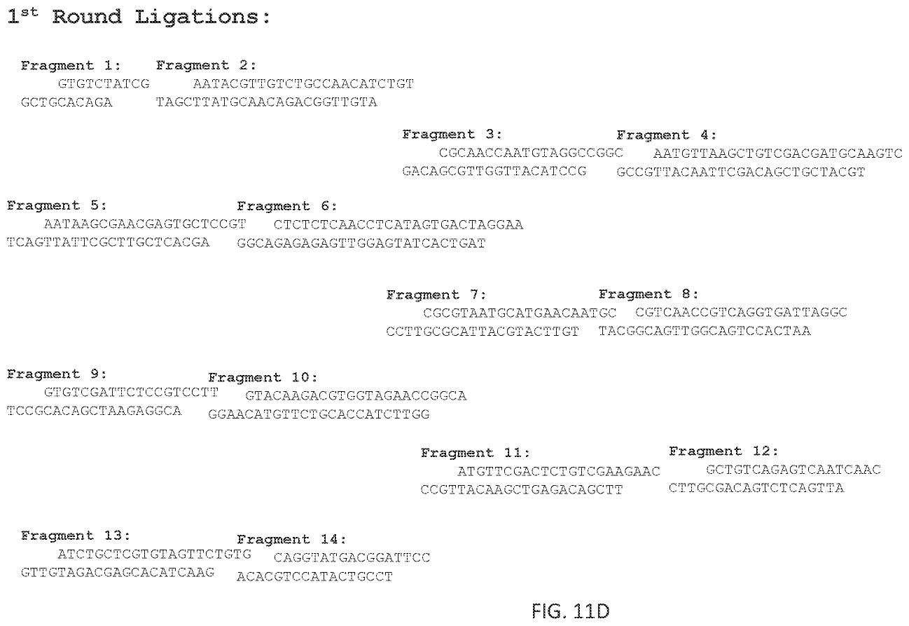

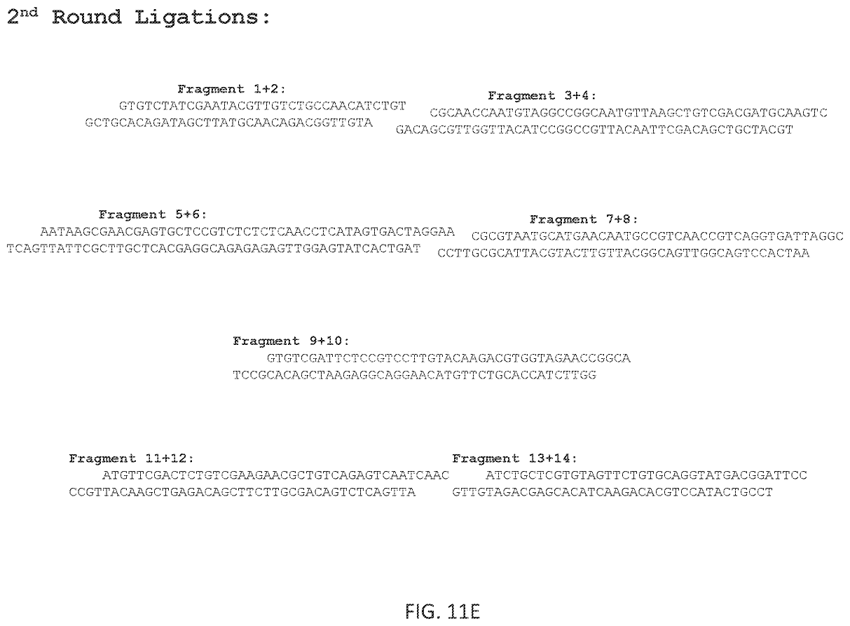

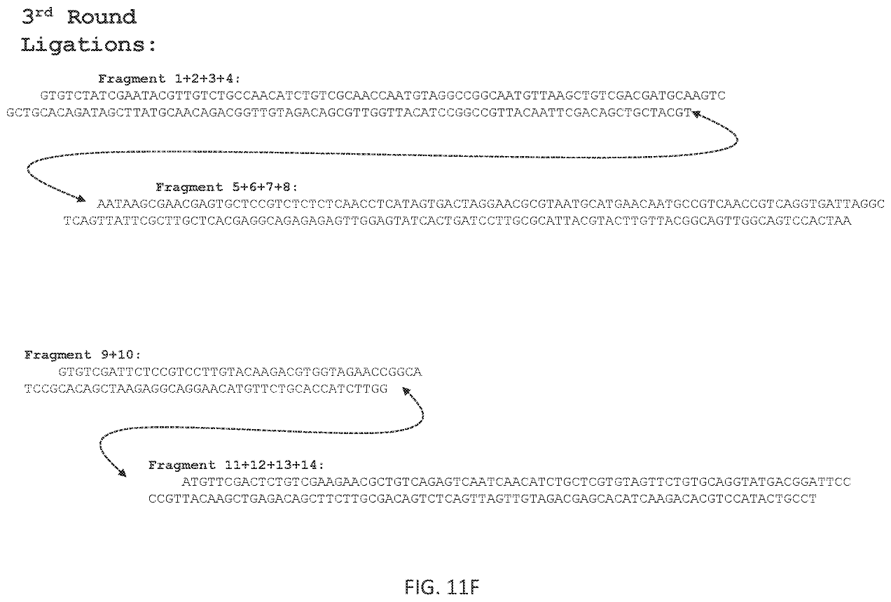

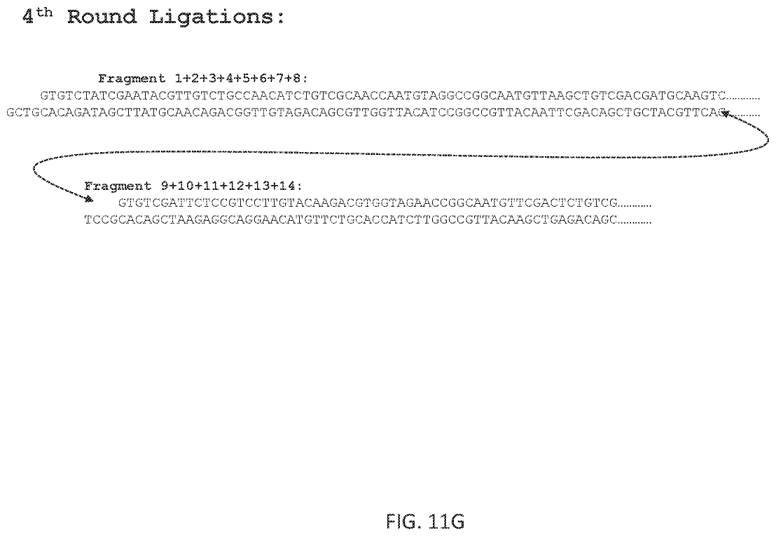

FIGS. 11A-11G illustrate a non-limiting example of a double-stranded gSynth assembly reaction. FIG. 11A shows a target sequence (entitled “5050Seq03”) that is to be synthesized using the double-stranded gSynth methods of the present disclosure. Parts of the sequence that are in bold and underlined correspond to 4-mer overhangs that have been selected, thus defining the fragments that will be used to synthesize the entire sequence. FIG. 11B shows the individual double-stranded nucleic acid fragments of the sequence shown in FIG. 11A that will be used in the double-stranded gSynth methods of the present disclosure to construct 5050Seq03. FIG. 11D is a schematic of the first round of ligations in the double-stranded gSynth method to synthesize the sequence shown in FIG. 11A. In the first ligation round, Fragments 1 and 2, Fragments 3 and 4, Fragments 5 and 6, Fragments 7 and 8, Fragments 9 and 10, Fragments 11 and 12, and Fragments 13 and 14 are hybridized via their complementary 5′ overhangs and then ligated together to create Fragment 1+2, Fragment 3+4, Fragment 5+6, Fragment 7+8, Fragment 9+10, Fragment 11+12, and Fragment 13+14. FIG. 1E is a schematic of the second round of ligations in the double-stranded gSynth method to synthesize the sequence shown in FIG. 11A. In the second ligation round, Fragments 1+2 and 3+4, Fragments 5+6 and 7+8, and Fragments 11+12 and 13+14 are hybridized via their complementary 5′ overhangs and then ligated together to create Fragment 1+2+3+4, Fragment 5+6+7+8, and Fragment 11+12+13+14. FIG. 1F is a schematic of the third round of ligations in the double-stranded gSynth method to synthesize the sequence shown in FIG. 11A. In the third ligation round, Fragments 1+2+3+4 and 5+6+7+8, and Fragments 9+10 and 11+12+13+14 are hybridized via their complementary 5′ overhangs and then ligated together to create Fragment 1+2+3+4+5+6+7+8 and Fragment 9+10+11+12+13+14. FIG. 1G is a schematic of the fourth and final round of ligations in the double-stranded gSynth method to synthesize the sequence shown in FIG. 11A. In the fourth ligation round, Fragments 1+2+3+4+5+6+7+8 and 9+10+11+12+13+14 are hybridized via their complementary 5′ overhangs and ligated together, thereby producing the sequence shown in FIG. 11A.

Accordingly, the pooled oligonucleotide synthesis methods and compositions disclosed herein can be used to produce the double-stranded fragments used in the gSynth methods described above and in PCT Application No. PCT/US2020/051838, published as WO2021055962A1.

### Additional Exemplary Embodiments

1. A double-stranded Addamer, wherein the Addamer comprises:
a) a first sequence allowing the generation of an overhang capable of ligation to complementary overhangs;
b) a payload sequence;
c) a second sequence allowing the generation of an overhangs capable of ligation to complementary overhangs; and
d) an Addamer, wherein at least one end of the Addamer comprises a hairpin structure.

2. The Addamer of claim 1, wherein the Addamer comprises a hairpin structure at both ends of the Addamer.

3. The Addamer of claim 1, wherein the first and second sequence allowing the generation of overhangs capable of ligation to complementary overhangs, are restriction endonuclease nickase recognition sites.

4. The Addamer of claim 1, wherein the first and second sequence allowing the generation of overhangs capable of ligation to complementary overhangs, are sites supporting the nicking activity of mutant Cas9 in the presence of sgRNA.

5. The Addamer of claim 1, wherein overhangs generated are 0, 1, 2, 3, 4 or 5 bases.

6. The Addamer of claim 1, wherein overhangs generated are 10 to 15 bases.

7. The Addamer of claim 1, wherein overhangs generated correspond to 4-base overhangs of a specific compatibility group.

8. A pool of oligonucleotides wherein each oligonucleotide is a strand of an Addamer but not directly complementary to any other oligonucleotide in the pool.

9. Two, several or many pools of oligonucleotides wherein each oligonucleotide is a strand of an Addamer wherein the mixture of any two, three, four or five pools uniquely leads to the formation of an Addamer or a group of Addamers comprising a gene or fragments of a gene.

10. A method for the generation of Addamer wherein the mixture of two or more pools of oligonucleotides under conditions favorable to hybridization leads to the formation of Addamer structures comprising the steps:
a) Hybridization
b) Ligation to resolve inherent nicks
c) Treatment with exonuclease to remove non-Addamer DNA

11. A method of claim 10, wherein the mixture is treated with His-tagged Taq MutS and Ni-NTA agarose to remove Addamers containing mismatched base pairs.

12. A method of gene assembly, wherein a group of Addamers with internal and external sites for digestion in a common volume are:
a) Treated with an endonuclease enzyme or set of enzymes or an enzyme combined with a short nucleic acid to remove one or both hairpins of the internal sites of each Addamer
b) Treated with ligase in the appropriate buffer
c) Treated with exonuclease to remove un-ligated material.

### EXAMPLES

### Example 1—Use of Pooled Oligonucleotides to Produce Addamers and Long Sequence Assemblies

In this example, pools (also referred to herein as pluralities) of oligonucleotides are used to generate a group of Addamers, and these Addamers are subsequently can be assembled into full-length target nucleic acid sequences.

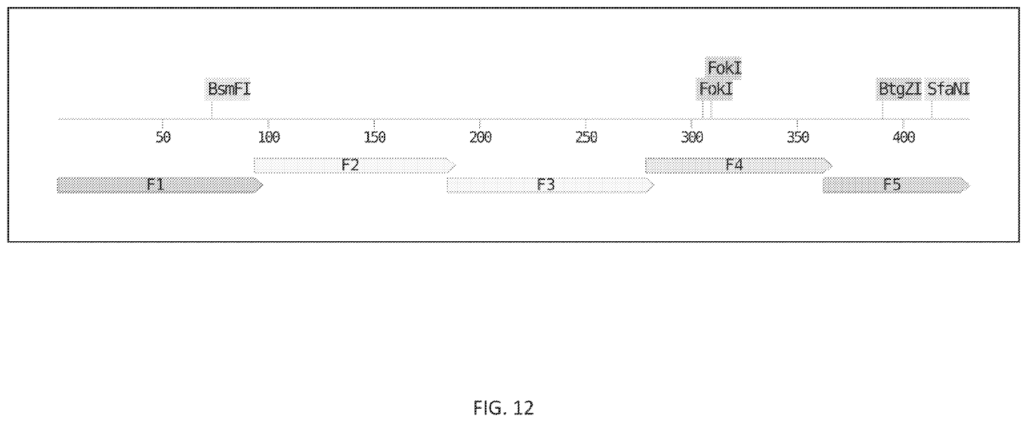

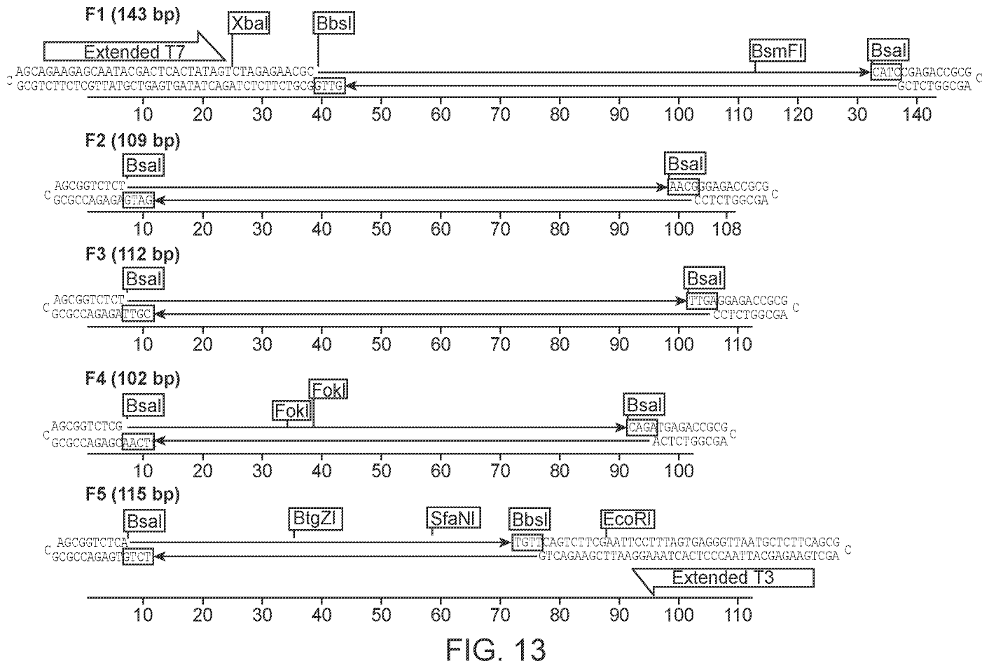

In this example, a graph theoretical method was used to programmatically divide a 431 bp sequence into five fragments (F1-F5), optimizing for approximately equal GC content across the fragments, and selecting fragments of about 100 bp in size (see FIG. 12). The predicted fragments had compatible single-stranded overhangs (CATC, AACG, TTGA, CAGA), with an estimated ligation fidelity of 100% (see FIG. 13).

Each predicted fragment sequence was used to create an Addamer that included Type IIS restriction endonuclease sites (IISREs) at either end of the payload. The end fragments F1 and F5 also contain PCR primer sites for amplification and restriction endonuclease (RE) sites for subsequent cloning steps (see FIG. 13).

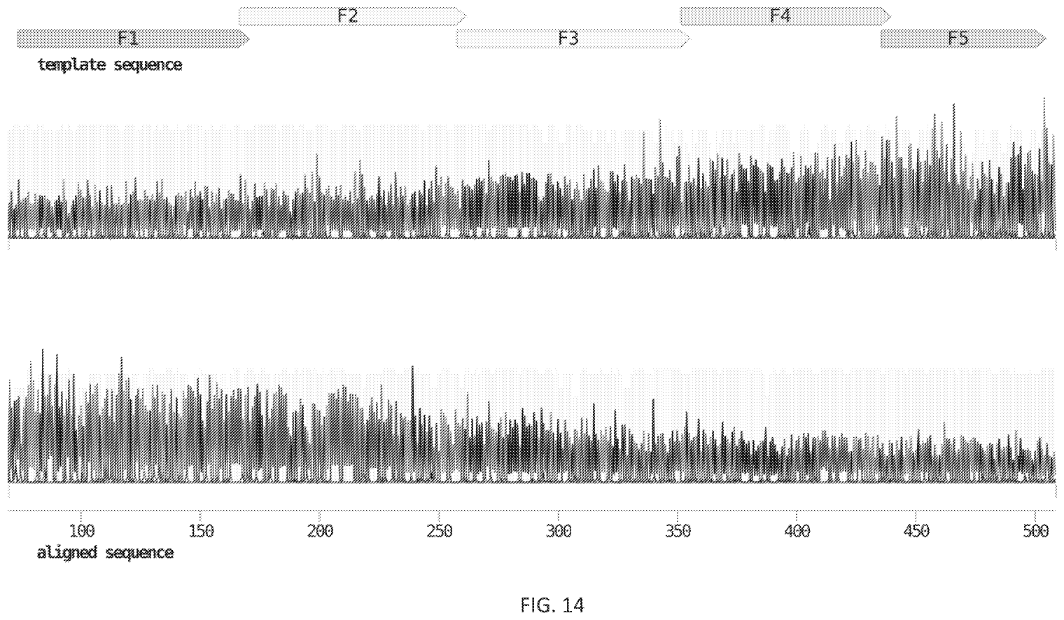

Each Addamer was formed from three oligonucleotide sequences provided from a pool (plurality) of nucleic acid molecules, as described herein. The Addamers were then assembled using the pooled synthesis method described in FIG. 6 and in further detail above. As shown in FIG. 6, IISREs was used to uncap each of the Addamers, leaving compatible 4 nucleotide single-stranded overhangs (in this example the IISRE is BsaI). The caps at the start of F1 and end of F5 remained intact, so that fragments will assemble into a single large Addamer, the product of the assembly. This larger Addamer sequence was designed to assemble in later steps into a longer sequence via the overhangs left by treatment with a different IISRE (in this example BbsI). The larger Addamer product was amplified, cut with REs XbaI and EcoRI and ligated into a cloning vector. Plasmid clones were subjected to capillary sequencing. Finally, it was confirmed that the assembled gene has the desired target sequence by aligning the results of capillary sequencing (see FIG. 14).

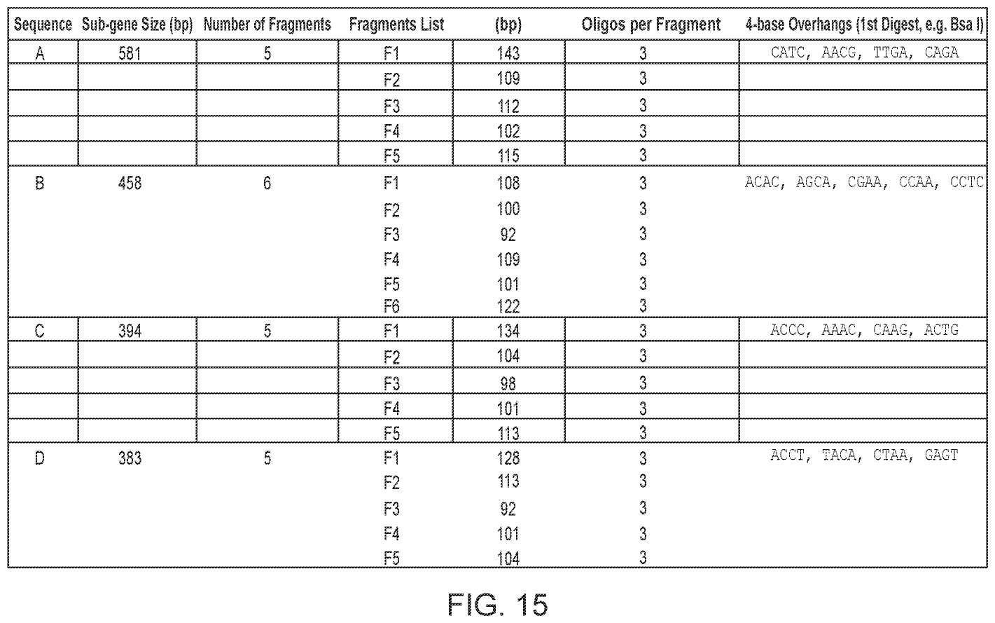

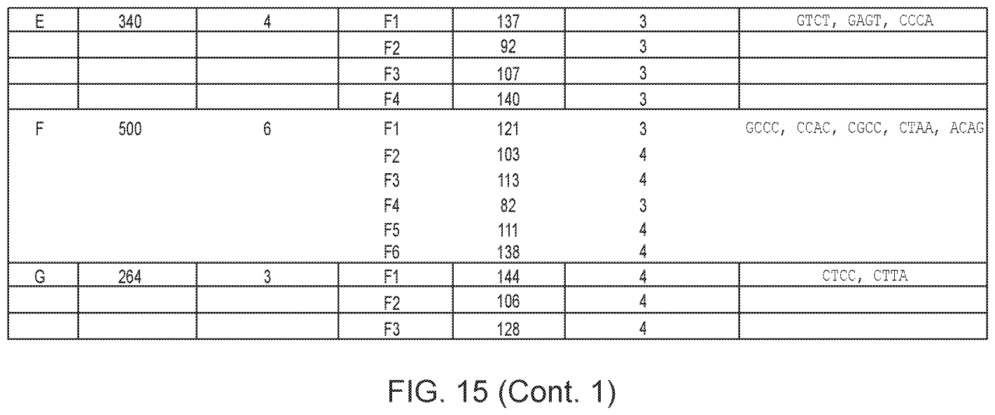

The protocol described above was also used to generate additional target sequences to demonstrate the generalizability of the method. The design of each of the assembly procedure for these additional target sequences are shown in FIG. 15 including the number of fragments get of the target sequences were subdivided into (the “Number of Fragment” column) as well as how many individual nucleic acid molecules were used to generate the Addmers that correspond to each fragment (the “Oligos per Fragment” column). These experiments demonstrate that that as many as six fragments can be combined to generate a larger sequence with Addamers. Additionally, the number of oligonucleotide sequences used to generate to component Addamers can be as many as four and can be used in a mixed pool of as many as 22 different oligonucleotide sequences simultaneously as is the case for Sequence F (see FIG. 15).

These results demonstrate that the compositions and methods described herein can be used to assembly and synthesize target nucleic acid sequences using an Addamer-based assembly method, wherein the individual Addamers are generated using pools (pluralities) of nucleic acid molecules produced using pooled oligonucleotide synthesis.

What is claimed is:

1. A composition comprising two or more pluralities of nucleic acid molecules,
wherein each of the pluralities of nucleic acid molecules comprises two or more species of nucleic acid molecules,
wherein different species of nucleic acid molecules comprise different nucleic acid sequences,
wherein within the two or more pluralities of nucleic acid molecules there is at least one set of corresponding pluralities such that when the at least one set of corresponding pluralities are combined in a single reaction volume, nucleic acid molecules from different pluralities within the set hybridize together to form at least one species of hybridized complex.

2. The composition of claim 1, wherein the composition comprises about:
a) six;
b) ten;
c) 15;
d) 20; or
e) 50
pluralities of nucleic acid molecules.

3. The composition of anyone of the preceding claims, wherein each plurality of nucleic acid molecules comprises at least about 25 different species of nucleic acid molecules.

4. The composition of anyone of the preceding claims, wherein each set of corresponding pluralities comprises the same number of pluralities.

5. The composition of anyone of the preceding claims, wherein the at least one species hybridized complex comprises one nucleic acid species from each of the pluralities within the set of corresponding pluralities.

6. The composition of anyone of the preceding claims, wherein the two or more pluralities of nucleic acid molecules are present in separate volumes.

7. The composition of anyone of the preceding claims, wherein the at least one set of corresponding pluralities comprises at least about:
a) two;
b) three;
c) four; or
d) five
pluralities of nucleic acid molecules.

8. The composition of anyone of the preceding claims, wherein the number of sets of corresponding pluralities is equal to:
$$X ! / Y ! ( X - Y ) !$$
wherein X is equal to the total number of pluralities of nucleic acid molecules, and Y is equal to the number of species of nucleic acid that hybridize together to form a single hybridized complex.

9. The composition of anyone of the preceding claims, wherein:
a) the at least one set of corresponding pluralities comprises two pluralities, and the at least one hybridized complex comprises two nucleic acid molecules;
b) the at least one set of corresponding pluralities comprises three pluralities, and the at least one hybridized complex comprises three nucleic acid molecules; or
c) the at least one set of corresponding pluralities comprises four pluralities, and the at least one hybridized complex comprises four nucleic acid molecules.

10. The composition of anyone of the preceding claims, wherein when a set of corresponding pluralities is combined in a single reaction, at least about five different hybridized complex species are formed.

11. The composition of anyone of the preceding claims, wherein the different species of nucleic acid molecules within a single plurality of nucleic acid molecules are not complementary to each other.

12. A method of producing at least one Addamer, the method comprising:
a) providing the composition of anyone of the preceding claims;
b) combining at least one set of corresponding pluralities of nucleic acid molecules in a single reaction volume such that at least one species of hybridized complex is formed;
c) contacting the at least one species of hybridized complex with at least one ligase enzyme to form that at least one Addamer capped at both ends by hairpins.

13. The method of claim 12, further comprising treating the products of step (c) with an exonuclease, thereby purifying properly ligated Addamers.

14. The method of claim 12 or claim 13, further comprising contacting the at least one species of hybridized complex with a MutS enzyme.

15. The method of any one of claims 12-14, wherein the Addamer comprises:
a) a first Type II S restriction endonuclease (IISRE) sequence;
b) a payload sequence;
c) an at least second IISRE sequence; and
wherein at least one end of the Addamer comprises a hairpin structure.

16. The method of any one of claims 12-15, wherein the Addamer comprises a hairpin structure at both ends of the Addamer.

17. The method of any one of claims 12-16, wherein the Addamer comprises:
a) a first IISRE sequence;
b) a second IISRE sequence;
c) a payload sequence; and
d) an at least third IISRE sequence.

18. The method of any one of claims 12-17, wherein the Addamer comprises:
a) a first IISRE sequence;
b) a second IISRE sequence;
c) a payload sequence;
d) a third IISRE sequence; and
e) an at least fourth IISRE sequence.

19. The method of any one of claims 12-18, wherein the Addamer further comprises a multiple cloning site (MCS) sequence, wherein the MCS sequence comprises one or more restriction endonuclease sequences.

20. The method of any one of claims 12-19, wherein at least one of the IISRE sequences are selected from a MlyI sequence, a NgoA VII sequence, SspD5I sequence, an AlwI sequence, a BccI sequence, a BcefI sequence, a PleI sequence, a BceAI sequence, a BceSIV sequence, a BscAI sequence, a BspD6I sequence, a FauI sequence, an EarI sequence, a BspQI sequence, a BfuAI sequence, a PaqCI sequence, an Esp3I sequence, a BbsI sequence, a BbvI sequence, a BtgZI sequence, a FokI sequence, a BsmFI sequence, a BsaI sequence, a BcoDI sequence and a HgaI sequence.

21. The method of any one of claims 12-20, wherein at least one hairpin structure comprises an aptamer sequence.

22. The method of any one of claims 12-21, wherein the aptamer sequence is selected from a pL1 aptamer sequence, a Thrombin 29-mer apatamer sequence, an S2.2 aptamer sequence, an ART1172 aptamer sequence, an R12.45 aptamer sequence, a Rb008 aptamer sequence and a 38NT SELEX apatamer sequence.

23. A method of synthesizing a nucleic acid molecule comprising a target nucleic acid sequence, the method comprising:
a) providing the composition of anyone of the preceding claims, wherein the composition comprises a set of corresponding pluralities of nucleic acid molecules such that when the set of corresponding pluralities are combined in a single reaction volume, nucleic acid molecules from different pluralities within the set hybridize together to form two or more species of hybridized complexes, wherein the two or more species of hybridized complexes comprises fragments of the target nucleic acid sequence;
b) combining the set of corresponding pluralities of nucleic acid molecules in a single reaction volume such that the two or more species of hybridized complexes are formed;
c) contacting the two or more species of hybridized complexes with at least one ligase enzyme to form two or more species of Addamers capped at both ends by hairpins;
d) assembling the two or more species of Addamers to synthesize the nucleic acid molecule comprising the target nucleic acid sequence.

24. The method of claim 23, wherein assembling the two or more species of Addamers comprises treating the Addamers with:
a) one or more restriction enzymes; and
b) one or more ligases,
either concurrently or sequentially, thereby assembling the nucleic acid molecule comprising the target nucleic acid sequence.

25. The method of claim 23, wherein assembling the two or more species of Addamers comprises treating the Addmers with:
a) modified Cas9 exhibiting nickase activity in combination with at least one guide RNA; and
b) one or more ligases,
either concurrently or sequentially, thereby assembling the nucleic acid molecule comprising the target nucleic acid sequence.


SEQUENCE LISTING (116 sequences)

SEQ ID NO: 1 - DNA, 19 bp, Artificial Sequence
  Synthetic Sequence
acagcgatgtctagatcgg

SEQ ID NO: 2 - DNA, 32 bp, Artificial Sequence
  Synthetic Sequence
cacgacctgcaaaaaggcactttttgcaggtc

SEQ ID NO: 3 - DNA, 36 bp, Artificial Sequence
  Synthetic Sequence
gttttccacgggaaaactgtcgctacagatctagcc

SEQ ID NO: 4 - DNA, 28 bp, Artificial Sequence
  Synthetic Sequence
cagcctttgacagcgatgtctagatcgg

SEQ ID NO: 5 - DNA, 18 bp, Artificial Sequence
  Synthetic Sequence
cacgacctgcaaaaaggc

SEQ ID NO: 6 - DNA, 27 bp, Artificial Sequence
  Synthetic Sequence
gggaaaactgtcgctacagatctagcc

SEQ ID NO: 7 - DNA, 17 bp, Artificial Sequence
  Synthetic Sequence
gtgctggacgtttttca

SEQ ID NO: 8 - DNA, 27 bp, Artificial Sequence
  Synthetic Sequence
gacatgagggtgcccgctcagctcctg

SEQ ID NO: 9 - DNA, 11 bp, Artificial Sequence
  Synthetic Sequence
gacatgagggt

SEQ ID NO: 10 - DNA, 11 bp, Artificial Sequence
  Synthetic Sequence
ctgtactccca

SEQ ID NO: 11 - DNA, 12 bp, Artificial Sequence
  Synthetic Sequence
gggtgcccgctc

SEQ ID NO: 12 - DNA, 12 bp, Artificial Sequence
  Synthetic Sequence
cccacgggcgag

SEQ ID NO: 13 - DNA, 12 bp, Artificial Sequence
  Synthetic Sequence
gctcagctcctg

SEQ ID NO: 14 - DNA, 12 bp, Artificial Sequence
  Synthetic Sequence
cgagtcgaggac

SEQ ID NO: 15 - DNA, 12 bp, Artificial Sequence
  Synthetic Sequence
gggtgcccgctc

SEQ ID NO: 16 - DNA, 12 bp, Artificial Sequence
  Synthetic Sequence
cccacgggcgag

SEQ ID NO: 17 - DNA, 12 bp, Artificial Sequence
  Synthetic Sequence
cccacgggcgag

SEQ ID NO: 18 - DNA, 12 bp, Artificial Sequence
  Synthetic Sequence
gctcagctcctg

SEQ ID NO: 19 - DNA, 12 bp, Artificial Sequence
  Synthetic Sequence
cgagtcgaggac

SEQ ID NO: 20 - DNA, 12 bp, Artificial Sequence
  Synthetic Sequence
gctcagctcctg

SEQ ID NO: 21 - DNA, 20 bp, Artificial Sequence
  Synthetic Sequence
gggtgcccgctcagctcctg

SEQ ID NO: 22 - DNA, 20 bp, Artificial Sequence
  Synthetic Sequence
cccacgggcgagtcgaggac

SEQ ID NO: 23 - DNA, 11 bp, Artificial Sequence
  Synthetic Sequence
gacatgagggt

SEQ ID NO: 24 - DNA, 11 bp, Artificial Sequence
  Synthetic Sequence
ctgtactccca

SEQ ID NO: 25 - DNA, 11 bp, Artificial Sequence
  Synthetic Sequence
ctgtactccca

SEQ ID NO: 26 - DNA, 20 bp, Artificial Sequence
  Synthetic Sequence
gggtgcccgctcagctcctg

SEQ ID NO: 27 - DNA, 20 bp, Artificial Sequence
  Synthetic Sequence
cccacgggcgagtcgaggac

SEQ ID NO: 28 - DNA, 20 bp, Artificial Sequence
  Synthetic Sequence
gggtgcccgctcagctcctg

SEQ ID NO: 29 - DNA, 16 bp, Artificial Sequence
  Synthetic Sequence
cgggcgagtcgaggac

SEQ ID NO: 30 - DNA, 27 bp, Artificial Sequence
  Synthetic Sequence
gacatgagggtgcccgctcagctcctg

SEQ ID NO: 31 - DNA, 27 bp, Artificial Sequence
  Synthetic Sequence
ctgtactcccacgggcgagtcgaggac

SEQ ID NO: 32 - DNA, 23 bp, Artificial Sequence
  Synthetic Sequence
gacatgagggtgcccgctcagct

SEQ ID NO: 33 - DNA, 23 bp, Artificial Sequence
  Synthetic Sequence
actcccacgggcgagtcgaggac

SEQ ID NO: 34 - DNA, 300 bp, Artificial Sequence
  Synthetic Sequence
cgacgtgtctatcgaatacgttgtctgccaacatctgtcgcaaccaatgtaggccggcaatgttaagctgtcgacgatgcaagtcaataagcgaacgagtgctccgtctctctcaacctcatagtgactaggaacgcgtaatgcatgaacaatgccgtcaaccgtcaggtgattaggcgtgtcgattctccgtccttgtacaagacgtggtagaaccggcaatgttcgactctgtcgaagaacgctgtcagagtcaatcaacatctgctcgtgtagttctgtgcaggtatgacggattcc

SEQ ID NO: 35 - DNA, 10 bp, Artificial Sequence
  Synthetic Sequence
gtgtctatcg

SEQ ID NO: 36 - DNA, 10 bp, Artificial Sequence
  Synthetic Sequence
gctgcacaga

SEQ ID NO: 37 - DNA, 24 bp, Artificial Sequence
  Synthetic Sequence
aatacgttgtctgccaacatctgt

SEQ ID NO: 38 - DNA, 24 bp, Artificial Sequence
  Synthetic Sequence
tagcttatgcaacagacggttgta

SEQ ID NO: 39 - DNA, 20 bp, Artificial Sequence
  Synthetic Sequence
cgcaaccaatgtaggccggc

SEQ ID NO: 40 - DNA, 20 bp, Artificial Sequence
  Synthetic Sequence
gacagcgttggttacatccg

SEQ ID NO: 41 - DNA, 27 bp, Artificial Sequence
  Synthetic Sequence
aatgttaagctgtcgacgatgcaagtc

SEQ ID NO: 42 - DNA, 27 bp, Artificial Sequence
  Synthetic Sequence
gccgttacaattcgacagctgctacgt

SEQ ID NO: 43 - DNA, 22 bp, Artificial Sequence
  Synthetic Sequence
aataagcgaacgagtgctccgt

SEQ ID NO: 44 - DNA, 22 bp, Artificial Sequence
  Synthetic Sequence
tcagttattcgcttgctcacga

SEQ ID NO: 45 - DNA, 27 bp, Artificial Sequence
  Synthetic Sequence
ctctctcaacctcatagtgactaggaa

SEQ ID NO: 46 - DNA, 27 bp, Artificial Sequence
  Synthetic Sequence
ggcagagagagttggagtatcactgat

SEQ ID NO: 47 - DNA, 21 bp, Artificial Sequence
  Synthetic Sequence
cgcgtaatgcatgaacaatgc

SEQ ID NO: 48 - DNA, 21 bp, Artificial Sequence
  Synthetic Sequence
ccttgcgcattacgtacttgt

SEQ ID NO: 49 - DNA, 23 bp, Artificial Sequence
  Synthetic Sequence
cgtcaaccgtcaggtgattaggc

SEQ ID NO: 50 - DNA, 23 bp, Artificial Sequence
  Synthetic Sequence
tacggcagttggcagtccactaa

SEQ ID NO: 51 - DNA, 19 bp, Artificial Sequence
  Synthetic Sequence
gtgtcgattctccgtcctt

SEQ ID NO: 52 - DNA, 19 bp, Artificial Sequence
  Synthetic Sequence
tccgcacagctaagaggca

SEQ ID NO: 53 - DNA, 24 bp, Artificial Sequence
  Synthetic Sequence
gtacaagacgtggtagaaccggca

SEQ ID NO: 54 - DNA, 24 bp, Artificial Sequence
  Synthetic Sequence
ggaacatgttctgcaccatcttgg

SEQ ID NO: 55 - DNA, 22 bp, Artificial Sequence
  Synthetic Sequence
atgttcgactctgtcgaagaac

SEQ ID NO: 56 - DNA, 22 bp, Artificial Sequence
  Synthetic Sequence
ccgttacaagctgagacagctt

SEQ ID NO: 57 - DNA, 19 bp, Artificial Sequence
  Synthetic Sequence
gctgtcagagtcaatcaac

SEQ ID NO: 58 - DNA, 19 bp, Artificial Sequence
  Synthetic Sequence
cttgcgacagtctcagtta

SEQ ID NO: 59 - DNA, 21 bp, Artificial Sequence
  Synthetic Sequence
atctgctcgtgtagttctgtg

SEQ ID NO: 60 - DNA, 21 bp, Artificial Sequence
  Synthetic Sequence
gttgtagacgagcacatcaag

SEQ ID NO: 61 - DNA, 17 bp, Artificial Sequence
  Synthetic Sequence
caggtatgacggattcc

SEQ ID NO: 62 - DNA, 17 bp, Artificial Sequence
  Synthetic Sequence
acacgtccatactgcct

SEQ ID NO: 63 - DNA, 34 bp, Artificial Sequence
  Synthetic Sequence
gtgtctatcgaatacgttgtctgccaacatctgt

SEQ ID NO: 64 - DNA, 34 bp, Artificial Sequence
  Synthetic Sequence
gctgcacagatagcttatgcaacagacggttgta

SEQ ID NO: 65 - DNA, 47 bp, Artificial Sequence
  Synthetic Sequence
cgcaaccaatgtaggccggcaatgttaagctgtcgacgatgcaagtc

SEQ ID NO: 66 - DNA, 47 bp, Artificial Sequence
  Synthetic Sequence
gacagcgttggttacatccggccgttacaattcgacagctgctacgt

SEQ ID NO: 67 - DNA, 49 bp, Artificial Sequence
  Synthetic Sequence
aataagcgaacgagtgctccgtctctctcaacctcatagtgactaggaa

SEQ ID NO: 68 - DNA, 49 bp, Artificial Sequence
  Synthetic Sequence
tcagttattcgcttgctcacgaggcagagagagttggagtatcactgat

SEQ ID NO: 69 - DNA, 44 bp, Artificial Sequence
  Synthetic Sequence
cgcgtaatgcatgaacaatgccgtcaaccgtcaggtgattaggc

SEQ ID NO: 70 - DNA, 44 bp, Artificial Sequence
  Synthetic Sequence
ccttgcgcattacgtacttgttacggcagttggcagtccactaa

SEQ ID NO: 71 - DNA, 43 bp, Artificial Sequence
  Synthetic Sequence
gtgtcgattctccgtccttgtacaagacgtggtagaaccggca

SEQ ID NO: 72 - DNA, 43 bp, Artificial Sequence
  Synthetic Sequence
tccgcacagctaagaggcaggaacatgttctgcaccatcttgg

SEQ ID NO: 73 - DNA, 41 bp, Artificial Sequence
  Synthetic Sequence
atgttcgactctgtcgaagaacgctgtcagagtcaatcaac

SEQ ID NO: 74 - DNA, 41 bp, Artificial Sequence
  Synthetic Sequence
ccgttacaagctgagacagcttcttgcgacagtctcagtta

SEQ ID NO: 75 - DNA, 38 bp, Artificial Sequence
  Synthetic Sequence
atctgctcgtgtagttctgtgcaggtatgacggattcc

SEQ ID NO: 76 - DNA, 38 bp, Artificial Sequence
  Synthetic Sequence
gttgtagacgagcacatcaagacacgtccatactgcct

SEQ ID NO: 77 - DNA, 81 bp, Artificial Sequence
  Synthetic Sequence
gtgtctatcgaatacgttgtctgccaacatctgtcgcaaccaatgtaggccggcaatgttaagctgtcgacgatgcaagtc

SEQ ID NO: 78 - DNA, 81 bp, Artificial Sequence
  Synthetic Sequence
gctgcacagatagcttatgcaacagacggttgtagacagcgttggttacatccggccgttacaattcgacagctgctacgt

SEQ ID NO: 79 - DNA, 93 bp, Artificial Sequence
  Synthetic Sequence
aataagcgaacgagtgctccgtctctctcaacctcatagtgactaggaacgcgtaatgcatgaacaatgccgtcaaccgtcaggtgattaggc

SEQ ID NO: 80 - DNA, 93 bp, Artificial Sequence
  Synthetic Sequence
tcagttattcgcttgctcacgaggcagagagagttggagtatcactgatccttgcgcattacgtacttgttacggcagttggcagtccactaa

SEQ ID NO: 81 - DNA, 43 bp, Artificial Sequence; identical to SEQ ID NO: 71
  Synthetic Sequence

SEQ ID NO: 82 - DNA, 43 bp, Artificial Sequence; identical to SEQ ID NO: 72
  Synthetic Sequence

SEQ ID NO: 83 - DNA, 79 bp, Artificial Sequence
  Synthetic Sequence
atgttcgactctgtcgaagaacgctgtcagagtcaatcaacatctgctcgtgtagttctgtgcaggtatgacggattcc

SEQ ID NO: 84 - DNA, 79 bp, Artificial Sequence
  Synthetic Sequence
ccgttacaagctgagacagcttcttgcgacagtctcagttagttgtagacgagcacatcaagacacgtccatactgcct

SEQ ID NO: 85 - DNA, 81 bp, Artificial Sequence; identical to SEQ ID NO: 77
  Synthetic Sequence

SEQ ID NO: 86 - DNA, 85 bp, Artificial Sequence
  Synthetic Sequence
gctgcacagatagcttatgcaacagacggttgtagacagcgttggttacatccggccgttacaattcgacagctgctacgttcag

SEQ ID NO: 87 - DNA, 59 bp, Artificial Sequence
  Synthetic Sequence
gtgtcgattctccgtccttgtacaagacgtggtagaaccggcaatgttcgactctgtcg

SEQ ID NO: 88 - DNA, 63 bp, Artificial Sequence
  Synthetic Sequence
tccgcacagctaagaggcaggaacatgttctgcaccatcttggccgttacaagctgagacagc

SEQ ID NO: 89 - DNA, 43 bp, Artificial Sequence
  Synthetic Sequence
cagcagaagagcaatacgactcactatagtctagagaagacgc

SEQ ID NO: 90 - DNA, 46 bp, Artificial Sequence
  Synthetic Sequence
gcgtcttctcgttatgctgagtgatatcagatctcttctgcggttg

SEQ ID NO: 91 - DNA, 15 bp, Artificial Sequence
  Synthetic Sequence
catccgagaccgcgc

SEQ ID NO: 92 - DNA, 10 bp, Artificial Sequence
  Synthetic Sequence
gctctggcga

SEQ ID NO: 93 - DNA, 11 bp, Artificial Sequence
  Synthetic Sequence
cagcggtctct

SEQ ID NO: 94 - DNA, 14 bp, Artificial Sequence
  Synthetic Sequence
gcgccagagagtag

SEQ ID NO: 95 - DNA, 15 bp, Artificial Sequence
  Synthetic Sequence
aacgggagaccgcgc

SEQ ID NO: 96 - DNA, 10 bp, Artificial Sequence
  Synthetic Sequence
cctctggcga

SEQ ID NO: 97 - DNA, 11 bp, Artificial Sequence
  Synthetic Sequence
cagcggtctct

SEQ ID NO: 98 - DNA, 14 bp, Artificial Sequence
  Synthetic Sequence
gcgccagagattgc

SEQ ID NO: 99 - DNA, 15 bp, Artificial Sequence
  Synthetic Sequence
ttgaggagaccgcgc

SEQ ID NO: 100 - DNA, 10 bp, Artificial Sequence
  Synthetic Sequence
cctctggcga

SEQ ID NO: 101 - DNA, 11 bp, Artificial Sequence
  Synthetic Sequence
cagcggtctcg

SEQ ID NO: 102 - DNA, 14 bp, Artificial Sequence
  Synthetic Sequence
gcgccagagcaact

SEQ ID NO: 103 - DNA, 15 bp, Artificial Sequence
  Synthetic Sequence
cagatgagaccgcgc

SEQ ID NO: 104 - DNA, 10 bp, Artificial Sequence
  Synthetic Sequence
actctggcga

SEQ ID NO: 105 - DNA, 11 bp, Artificial Sequence
  Synthetic Sequence
cagcggtctca

SEQ ID NO: 106 - DNA, 14 bp, Artificial Sequence
  Synthetic Sequence
gcgccagagtgtct

SEQ ID NO: 107 - DNA, 47 bp, Artificial Sequence
  Synthetic Sequence
tgttcagtcttcgaattcctttagtgagggttaatgctcttcagcgc

SEQ ID NO: 108 - DNA, 42 bp, Artificial Sequence
  Synthetic Sequence
gtcagaagcttaaggaaatcactcccaattacgagaagtcga

SEQ ID NO: 109 - DNA, 32 bp, Artificial Sequence
  Synthetic Sequence
tcgattggattgtgccggaagtgctggctcga

SEQ ID NO: 110 - DNA, 29 bp, Artificial Sequence
  Synthetic Sequence
agtccgtggtagggcaggttggggtgact

SEQ ID NO: 111 - DNA, 23 bp, Artificial Sequence
  Synthetic Sequence
cagttgatcctttggataccctg

SEQ ID NO: 112 - DNA, 42 bp, Artificial Sequence
  Synthetic Sequence
ggcgtgcagtgccttcggccgtgcggtgcctccgtcacgcct

SEQ ID NO: 113 - DNA, 43 bp, Artificial Sequence
  Synthetic Sequence
accgtctgagcgattcgtactttattcgggaggtatcagcggg

SEQ ID NO: 114 - DNA, 34 bp, Artificial Sequence
  Synthetic Sequence
cctggacggaaccagaatacttttggtctccagg

SEQ ID NO: 115 - DNA, 43 bp, Artificial Sequence
  Synthetic Sequence
aaatacccccccttcggtgcaaagcaccgaagggggggtattt

SEQ ID NO: 116 - DNA, 11 bp, Artificial Sequence
  Synthetic Sequence
gacatgagggt